(19) United States
(12) Patent Application Publication
(10) Pub. No.: US 2022/0273591 A1
(43) Pub. Date: Sep. 1, 2022

(54) METHODS FOR DETERMINING RISK OF DEVELOPING INSULIN RESISTANCE

(71) Applicant: The Board of Trustees of the Leland Stanford Junior University, Stanford, CA (US)

(72) Inventors: Joseph C. Wu, Stanford, CA (US); Edward Lau, Redwood City, CA (US); Mark Chandy, Redwood City, CA (US)

(21) Appl. No.: 17/629,571

(22) PCT Filed: Jul. 24, 2020
(86) PCT No.: PCT/US2020/043546

§ 371 Date: Jan. 24, 2022

Related U.S. Application Data
(60) Provisional application No. 62/879,237, filed on Jul. 26, 2019.

(51) Int. Cl. A61K 31/155; C12N 5/071; C12Q 1/6883; G01N 33/68; G01N 33/50; A61K 38/26; A61K 31/64; A61K 31/426; A61P 3/10; G16H 50/30

(52) U.S. Cl. CPC A61K 31/155; C12N 5/0692; C12Q 1/6883; G01N 33/689; G01N 33/5064; A61K 38/26; A61K 31/64; A61K 31/426; A61P 3/10; G16H 50/30; C12N 2506/45; C12N 2501/25; C12Q 2600/118; C12Q 2600/158; C12Q 1/6851

(57) ABSTRACT

Compositions, methods, and kits are provided for determining whether a subject is at risk of developing insulin resistance. In particular, phosphorylated Akt, reactive oxygen species (ROS), SIRT1, eNOS, CDH13, IRS1 and NO production have been identified as biomarkers associated with insulin resistance and type 2 diabetes. The diagnostic methods comprise measuring the level of at least one biomarker in induced pluripotent stem cells derived from somatic cells of the subject, which have been differentiated into endothelial cells (IPSC-ECs).

### BACKGROUND OF THE INVENTION

Type 2 diabetes (T2DM) is a global epidemic affecting over 400 million people and is a leading cause of morbidity and mortality (Roglic (2016) Int J Noncommun Dis. 1(2):65-75). The prevalence of disease is increasing as obesity and sedentary lifestyle are becoming more common in developing countries. T2DM has a strong association with cardiovascular disease (CVD)—the number one cause of death globally, with 17.5 million deaths per year (Haffner et al. (1998) N Engl J Med. 339:229-234, Health Organization W. Global status report on noncommunicable diseases 2014, apps.who.int/iris/bitstream/handle/10665/148114/).

Insulin resistance is associated with obesity and metabolic syndrome and is central to the development of T2DM (Reaven (1988) Diabetes 37:1595-1607). A risk factor for CVD, insulin resistance is associated with an increased risk of CVD death (Malik et al. (2004) Circulation. 110:1245-1250, Reaven (2012) Arterioscler Thromb Vasc Biol. 32:1754-1759). Often underdiagnosed (Donner et al. (1985) J Clin Endocrinol Metab. 60:723-726.), the prevalence of insulin resistance is estimated to be three times greater than T2DM (Meigs (2003) Curr Diab Rep. 3:73-79). Along with the diagnostic challenge, the treatment of insulin resistance is limited to diet and exercise with metformin being the only drug known to delay onset (Knowler et al. (2002) N Engl J Med. 346:393-403). The causes of insulin resistance and effects on the vascular system are not well understood and consequently therapy is limited.

There remains a need for better methods of assessing the risk of developing insulin resistance and T2DM.

### SUMMARY OF THE INVENTION

Compositions, methods, and kits for analysis of an individual's risk of developing insulin resistance and methods of treatment based on such analysis are provided. In particular, phosphorylated Akt, reactive oxygen species (ROS), nitric oxide (NO), SIRT1, eNOS, CDH13, and IRS1 are analyzed to provide a measurement of the risk of insulin resistance. The levels of one or more of these biomarkers are measured in induced pluripotent stem cells derived from somatic cells of the subject, which have been differentiated into endothelial cells (IPSC-ECs). Determining whether the IPSC-ECs derived from a patient are insulin-resistant or insulin-sensitive predicts the risk of developing insulin resistance.

In one aspect, the presence of phosphorylated Akt, ROS, NO, SIRT1, eNOS, CDH13, and IRS1 is analyzed, and their levels are utilized to provide an evaluation of risk for developing insulin resistance.

In another aspect, a method of analysis for risk of developing insulin resistance is provided, the method comprising: a) obtaining a biological sample comprising somatic cells from the subject; b) generating Induced pluripotent stem cells (IPSCs) from the somatic cells; c) differentiating the IPSCs into endothelial cells (IPSC-ECs); d) measuring one or more biomarkers in the IPSC-ECs derived from the subject, wherein the biomarkers are selected from the group consisting of phosphorylated Akt, ROS, NO, SIRT1, eNOS, CDH13, and IRS1; and e) determining whether an individual is at risk of developing insulin resistance, wherein reduced phosphorylation of Akt, increased levels of ROS, decreased levels of NO, decreased levels of expression of SIRT1 and IRS1, and increased levels of expression of eNOS and CDH13 in the IPSC-ECs derived from the subject compared to reference values ranges for levels of the biomarkers in control IPSC-ECs indicate the subject is at risk of developing insulin resistance.

The method may be performed, for example, on a sample from a subject who has not yet developed the pathological changes that cause clinical signs and symptoms of pre-diabetes or diabetes and consequent vascular disease. In particular, the methods described herein are useful for monitoring an individual who has known risk factors for developing insulin resistance and/or type 2 diabetes, such as, but not limited to, obesity, inadequate exercise (i.e., sedentary lifestyle), a genetic predisposition for developing diabetes, a family history indicating a risk of developing diabetes (e.g., first degree relatives with diabetes), a disease associated with insulin resistance (e.g., polycystic ovary syndrome, hepatitis C), high blood pressure, high cholesterol, age of 45 or older, history of gestational diabetes, vitamin D deficiency, treatment with HIV drugs having protease inhibitors, or growth hormone replacement therapy. The method can be performed, for example, on samples from children with a strong family history indicating a risk of developing insulin resistance or type 2 diabetes, wherein if a child is identified as being at risk of developing insulin resistance, lifestyle changes (e.g., diet modification, weight control, adequate exercise, etc., as discussed further below) can be recommended early in life to minimize the risk and potentially avoid the pathological physiological changes leading to insulin resistance or type 2 diabetes.

In certain embodiments, the method further comprises administering a pre-diabetic treatment if the subject is determined to be at risk of developing insulin resistance. The pre-diabetic treatment may comprise, for example, without limitation, administering a therapeutically effective amount of metformin to the subject, modifying diet of the subject to reduce carbohydrate intake, reducing weight of the subject, increasing amount of exercise of the subject, bariatric surgery, administering medication to treat high blood pressure in the subject, administering medication to treat high cholesterol in the subject, ceasing smoking of the subject, or a combination thereof. In some embodiments, the treatment further comprises administering to the subject a sulfonylurea, a thiazolidinedione, a dipeptidyl peptidase-4 inhibitor, a sodium-glucose transport protein 2 (SGLT2) inhibitor, a glucagon-like peptide-1 analog, an angiotensin-converting enzyme inhibitor (ACEI), or a combination thereof.

In certain embodiments, the subject is a human being. In some embodiments, the subject is obese. In some embodiments, the subject is a South Asian subject.

The somatic cells may include, for example, without limitation, peripheral blood mononuclear cells (PBMCs), fibroblasts, keratinocytes, epithelial cells, or endothelial progenitor cells.

In certain embodiments, measuring the level of expression of SIRT1, eNOS, CDH13, or IRS1 comprises measuring a level of expression of an mRNA or protein. In some embodiments, the level of an mRNA is measured by performing a hybridization-based method, a polymerase chain reaction (PCR)-based method, or a nucleic acid sequencing method. For example, a mRNA may be measured by a method including, without limitation, microarray analysis, polymerase chain reaction (PCR), reverse transcriptase polymerase chain reaction (RT-PCR), Northern blot, RNA-Seq, or serial analysis of gene expression (SAGE). In other embodiments, measuring a level of a biomarker protein comprises performing mass spectrometry or tandem mass spectrometry, an enzymatic or biochemical assay, liquid chromatography, NMR, or an immunoassay, including, without limitation, an enzyme-linked immunosorbent assay (ELISA), a radioimmunoassay (RIA), an immunofluorescent assay (IFA), fluorescence-activated cell sorting (FACS), or a Western Blot.

In another aspect, a method of monitoring progression of prediabetes in a subject is provided, the method comprising: a) obtaining a first biological sample comprising somatic cells from the subject at a first time point and a second biological sample comprising somatic cells from the subject later at a second time point; b) generating induced pluripotent stem cells (IPSCs) from the somatic cells in the first biological sample and the second biological sample; c) differentiating the IPSCs derived from the somatic cells of the first biological sample and the second biological sample into IPSC-ECs; d) measuring one or more biomarkers in the IPSC-ECs derived from the somatic cells of the first biological sample and the second biological sample, wherein the biomarkers are selected from the group consisting of phosphorylated Akt, ROS, SIRT1, eNOS, CDH13, and IRS1; and e) evaluating progression of prediabetes in the subject wherein detection of reduced phosphorylation of Akt, increased levels of ROS, decreased levels of NO, decreased levels of expression of SIRT1 and IRS1, and increased levels of expression of eNOS and CDH13 in the IPSC-ECs derived from the somatic cells of the second biological sample compared to the IPSC-ECs derived from the somatic cells of the first biological sample indicates that the risk of the subject developing insulin resistance is increasing, and detection of increased phosphorylation of Akt, decreased levels of ROS, increased levels of NO, increased levels of expression of SIRT1 and IRS1, and decreased levels of expression of eNOS and CDH13 in the IPSC-ECs derived from the somatic cells of the second biological sample compared to the IPSC-ECs derived from the somatic cells of the first biological sample indicates that the risk of the subject developing insulin resistance is decreasing. In some embodiments, the above biomarkers may be measured after treating IPSC-ECs with a stimulation cocktail including but not limited to glucose and TNF-alpha to simulate the extracellular milieu in prediabetic or diabetic patients to illicit phenotypic differences.

In some embodiments, the subject has developed clinical symptoms.

In some embodiments, the method further comprises administering a pre-diabetic treatment to the subject before the second time point such as, but not limited to, administration of metformin to the subject, modifying diet of the subject to reducing carbohydrate intake of the subject, reducing weight of the subject, increasing amount of exercise of the subject, bariatric surgery, administering medication to treat high blood pressure in the subject, administering medication to treat high cholesterol in the subject, ceasing smoking of the subject, or a combination thereof. In some embodiments, the treatment further comprises administering to the subject a sulfonylurea, a thiazolidinedione, a dipeptidyl peptidase-4 inhibitor, a sodium-glucose transport protein 2 (SGLT2) inhibitor, a glucagon-like peptide-1 analog, an angiotensin-converting enzyme inhibitor (ACEI), or a combination thereof.

In another aspect, a method of monitoring efficacy of a pre-diabetic treatment in a subject is provided, the method comprising: a) obtaining a first biological sample comprising somatic cells from the subject before the subject undergoes the pre-diabetic treatment and a second biological sample comprising somatic cells from the subject after the subject undergoes the pre-diabetic treatment; b) generating IPSCs from the somatic cells in the first biological sample and the second biological sample; c) differentiating the IPSCs derived from the somatic cells of the first biological sample and the second biological sample into IPSC-ECs; d) measuring one or more biomarkers in the IPSC-ECs derived from the somatic cells of the first biological sample and the second biological sample, wherein the biomarkers are selected from the group consisting of phosphorylated Akt, ROS, NO, SIRT1, eNOS, CDH13, and IRS1; and e) evaluating the efficacy of the prediabetic treatment, wherein detection of reduced phosphorylation of Akt, increased levels of ROS, decreased levels of NO release in the presence or absence of stimuli, decreased levels of expression of SIRT1 and IRS1, and increased levels of expression of eNOS and CDH13 in the IPSC-ECs derived from the somatic cells of the second biological sample compared to the IPSC-ECs derived from the somatic cells of the first biological sample indicates that the subject is worsening or not responding to the pre-diabetic treatment, and detection of increased phosphorylation of Akt, decreased levels of ROS, increased levels of NO, increased levels of expression of SIRT1 and IRS1, and decreased levels of expression of eNOS and CDH13 in the IPSC-ECs derived from the somatic cells of the second biological sample compared to the IPSC-ECs derived from the somatic cells of the first biological sample indicates that the subject is improving.

In certain embodiments, the subject has developed clinical symptoms.

In certain embodiments, the pre-diabetic treatment comprises administering a therapeutically effective amount of metformin to the subject, modifying diet of the subject to reduce carbohydrate intake, reducing weight of the subject, increasing amount of exercise of the subject, bariatric surgery, administering medication to treat high blood pressure in the subject, administering medication to treat high cholesterol in the subject, ceasing smoking of the subject, or a combination thereof. In some embodiments, the treatment further comprises administering to the subject a sulfonylurea, a thiazolidinedione, a dipeptidyl peptidase-4 inhibitor, a sodium-glucose transport protein 2 (SGLT2) inhibitor, a glucagon-like peptide-1 analog, an angiotensin-converting enzyme inhibitor (ACEI), or a combination thereof.

In certain embodiments, the method further comprises altering the pre-diabetic treatment if the subject is worsening or not responding to the pre-diabetic treatment. For example, a pre-diabetic treatment may be altered in various ways, including, without limitation, by increasing exercise of the subject, increasing weight loss of the subject, e.g., with a stricter diet or bariatric surgery, administering metformin to the subject, or any combination thereof.

In another aspect, a kit for determining whether an individual is at risk of developing insulin resistance is provided, the kit comprising reagents for generating IPSC-ECs from somatic cells of a subject (e.g., Yamanaka factors, vascular endothelial growth factor (VEGF), basic fibroblast growth factor (bFGF), and bone morphogenetic protein 4 (BMP4); and means for detecting one or more biomarkers selected from the group consisting of phosphorylated Akt, ROS, NO, SIRT1, eNOS, CDH13, and IRS1. In some embodiments, the kit further comprises instructions for determining whether an individual is at risk of developing insulin resistance.

In another aspect, a model of insulin resistance is provided comprising an induced pluripotent stem cell-derived endothelial cell (IPSC-EC), wherein the IPSC-EC is derived from a somatic cell of a subject and treated with tumor necrosis factor-alpha (TNFα), interleukin-1 beta (IL1-β), and interferon gamma (INFγ), as described herein. This model may utilize, in a non-limiting fashion, one or more simple or multiple linear regression models, general additive models, mixed models, generalized linear models or machine learning methods to derive a IPSC-EC-based risk score for insulin resistance.

### BRIEF DESCRIPTION OF THE DRAWINGS

FIGS. 1A-1D. Patient demographics reveal no difference in age or sex, but insulin-resistant patients have a higher BMI. FIG. 1A. Baseline demographics of healthy control (n=5) and insulin-resistant patients (n=5). No significant difference was noted for age, sex and fasting glucose. BMI (p=0.03) and insulin AUC (p=0.003) were elevated in insulin-resistant participants. Data represents median and interquartile range. FIG. 1B. Peripheral arterial tonometry in the occluded arm and control arm of health control (HC) and insulin resistant (IR) participants, FIG. 1C. Reactive hyperemia index (RHI) of HC and IR participants. IR participants had a lower RHI compared to HC (p=0.03). FIG. 1D. Heart rate of HC and IR patients subjected to peripheral artery tonometry. Participants had no significant difference in HR between these two groups.

FIGS. 2A-2D. Insulin-resistant iPSC-derived endothelial cells are dysfunctional at baseline. FIG. 2A. Tube formation assays of healthy control (upper) and insulin-resistant (lower) cells lines. For each like, iPSC-EC were treated with vehicle or prediabetic cocktail consisting of 12.5 mM glucose and 5 ng TNFα. FIG. 2B. The covered areas for each cell lines. Micrographs were analyzed on Image J. FIG. 2C. Total nitric oxide production of insulin resistance and healthy control lines treated with vehicle or prediabetic cocktail at baseline and with insulin. FIG. 2D. The amount of induced NO release by insulin over baseline with healthy control and insulin-resistant iPSC-EC under vehicle (left) or the prediabetic cocktail (right).

FIGS. 3A-3B. Insulin signaling is impaired and expression profiles of genes implicated in metabolic dysfunction are perturbed in resistant iPSC-EC lines. FIG. 3A. Immunoblot of whole cell extract produced from healthy control and insulin resistant iPSC-ECs. Cells from each group were treated with either vehicle, 100 nM insulin or 100 nM insulin and 5 ng/mL TNFα. eNOS expression and phosphorylation is increased at baseline and insulin stimulated phosphorylation is attenuated in insulin resistant lines. TNFα decreases expression of eNOS and further attenuates insulin mediated phosphorylation. (right) Densitometry confirms the increased eNOS phosphorylation in healthy control lines relative to insulin resistant lines after insulin stimulation. FIG. 3B. The expression profile of genes implicated in metabolic dysfunction. The expression of in healthy control and insulin resistant iPSC-EC are similar except CDH13 and NOS3 expression are increased in IR lines although not significantly. However, IRS1 gene expression is significantly reduced in IR lines (p=0.028).

FIGS. 4A-4F. Multiple linear regression of iPSC-ECs cellular phenotypes predict insulin resistance risk. (FIG. 4A) Correlation matrix of model parameters. First we wanted to visualize all the measured parameters (age, BMI, fasting glucose, insulin release, eNOS/IRS/CDH13 expression, insulin-mediated iPSC-ECs and NO release under baseline and stimulated conditions) and determine whether they are correlated. FIG. 4B. First we wanted to know how well we can predict insulin AUC by clinical parameters (age, BMI, fasting glucose); sex was not included because it was invariant among the subjects. All three parameters moderately predict insulin area under the curve (AUC) which is a measurement for insulin resistance with an R² of 0.696 FIG. 4C. Next, we did a comparison with three iPSC-ECs gene expression data (NOS3, IRS1, and CDH13 qPCR data) to see if they can predict patient insulin AUC. Gene expression of CDH13, IRS1 and NOS3 have high correlation (r² of 0.858) with insulin AUC. FIG. 4D. Demographic profile of diabetic patients recruited from the Stanford Cardiovascular Institute (SCVI) biobank. FIG. 4E. Gene expression profile of CDH13, NOS3 and IRS1 are not perturbed significantly in insulin resistant iPSC-EC. FIG. 4F. The gene expression algorithm built on insulin resistance predicts elevated insulin AUC.

FIGS. 5A-5E. Characterization of iPSC-derived endothelial cells. FIG. 5A. The protocol for iPSC-EC differentiation. FIG. 5B. Micrographs of iPSC-ECs differentiated from healthy control (HC) and insulin-resistant (IR) donor iPSC lines. FIG. 5C. Real time PCR analysis reveals that iPSC-ECsexpress CD31 and CD144 when compared to iSPC. Label: P values of two-tailed t-test. FIG. 5D. Representative flow cytometry of iPSC-ECs form healthy control and insulin resistant lines. FIG. 5E. Immunostaining for eNOS and CHD13 in iPSC-endothelial cells. A representative healthy control and insulin-resistant iPSC-ECsline were stained with anti-eNOS and anti-CHD13.

FIGS. 6A-6E. South Asian insulin-resistant patients have unique iPSC derived endothelial cellular and molecular biomarkers. FIG. 6A. Cytokines induced expression of cell surface adhesion molecules in iPSC-derived endothelial cells. The expression of cell surface adhesion molecules (ICAM1 and VCAM) is increased in iPSC-EC in response to interleukin 1β (IL-1β), interferon γ (INFγ) and TNFα treatment. FIG. 6B. TNFα treatment does not induce endothelial dysfunction. iPSC-EC were treated with 10 ng/ml TNFα and expression of CD31 and CD144 were not affected; however, expression of cell surface adhesion molecule ICAM1 was significantly increase (p=8.6×10⁶). FIG. 6C. A representative immunoblot showing that healthy control iPSC-ECs have increased Akt and eNOS phosphorylation in response to insulin. TNFα disrupts insulin signaling. At baseline, insulin-resistant lines have increased eNOS expression and also increased phosphorylated eNOS at baseline and there is no increase in phosphorylated eNOS expression with insulin, when compared to healthy controls. TNFα disrupts eNOS expression and phosphorylation. FIG. 6D. Real time quantitative PCR reveals that SIRT1 is decreased in iPSC-EC insulin resistant lines. FIG. 6E. iPSC-EC display decreased tube formation in the presence of 10 ng/ml TNFα.

FIGS. 7A-7D. Endothelial function in insulin resistant iPSC-ECs. FIG. 7A. Tube length (μM0 distribution and FIG. 7B. branch points in tube formation assays, analyzed using ImageJ. FIG. 7C. Wound healing assay shows no significant differences between insulin-resistant and healthy controls iPSC-ECs under 10 ng/mL TNFα. FIG. 7D. Flow cytometry of DAF-FM stained iPSC-ECs from healthy and insulin-resistant lines.

FIGS. 8A-8B. Adiponectin receptor 3 (CDH13) is upregulated in insulin-resistant cell lines and augments eNOS expression while diminishing insulin sensitivity. CHD13 expression is upregulated when assayed by real time PCR (FIG. 8A) or immunoblots (FIG. 8B) in insulin-resistant lines. CDH13 siRNA experiments reveal that CDH13 expression is reduced by more than 85% resulting in decreased eNOS expression to fall and IRS1 expression to increase in healthy control (C) and insulin-resistant line (D).

### DETAILED DESCRIPTION OF THE INVENTION

Compositions, methods, and kits are provided for determining whether a subject is at risk of developing insulin resistance. In particular, phosphorylated Akt, reactive oxygen species (ROS), NO, SIRT1, eNOS, CDH13, and IRS1 have been identified as biomarkers associated with insulin resistance and type 2 diabetes. The diagnostic methods comprise measuring the level of at least one biomarker in induced pluripotent stem cells derived from somatic cells of the subject, which have been differentiated into endothelial cells (IPSC-ECs).

Before the present compositions, methods, and kits are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a biomarker” includes a plurality of such biomarkers and reference to “the polypeptide” includes reference to one or more polypeptides and equivalents thereof, e.g. peptides or proteins known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

Biomarkers. The term “biomarker” as used herein refers to a compound, such as a protein, a mRNA, a metabolite, or a metabolic byproduct which is differentially expressed or present at different concentrations, levels or frequencies in one sample compared to another, such as a sample of IPSC-ECs derived from patients who are insulin resistant or who have type 2 diabetes compared to IPSC-ECs derived from healthy control subjects (i.e., subjects not having diabetes or insulin resistance). Biomarkers include, but are not limited to, phosphorylated Akt, reactive oxygen species (ROS), NO, SIRT1, eNOS, CDH13, and IRS1.

In some embodiments, the concentration or level of a biomarker is determined before and after the administration of a pre-diabetic treatment to a subject. The pre-diabetic treatment may comprise, for example, without limitation, administering a therapeutically effective amount of metformin to the subject, modifying diet of the subject to reduce carbohydrate intake, reducing weight of the subject, increasing amount of exercise of the subject, bariatric surgery, administering medication to treat high blood pressure in the subject, administering medication to treat high cholesterol in the subject, ceasing smoking of the subject, or a combination thereof. In some embodiments, the treatment further comprises administering to the subject a sulfonylurea, a thiazolidinedione, a dipeptidyl peptidase-4 inhibitor, a sodium-glucose transport protein 2 (SGLT2) inhibitor, a glucagon-like peptide-1 analog, an angiotensin-converting enzyme inhibitor (ACEI), or a combination thereof. The degree of change in the concentration or level of a biomarker, or lack thereof, is interpreted as an indication of whether the pre-diabetic treatment has the desired effect (e.g., lowering the risk of the patient developing insulin resistance and/or disease progression to type 2 diabetes). In other words, the concentration or level of a biomarker is determined before and after the administration of the pre-diabetic treatment to an individual, and the degree of change, or lack thereof, in the level is interpreted as an indication of whether the individual is “responsive” to the treatment.

A “reference level” or “reference value” of a biomarker means a level of the biomarker that is indicative of a particular disease state, phenotype, or predisposition to developing a particular disease state or phenotype, or lack thereof, as well as combinations of disease states, phenotypes, or predisposition to developing a particular disease state or phenotype, or lack thereof. A “positive” reference level of a biomarker means a level that is indicative of a particular disease state or phenotype. A “negative” reference level of a biomarker means a level that is indicative of a lack of a particular disease state or phenotype. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched or gender-matched so that comparisons may be made between biomarker levels in samples from subjects of a certain age or gender and reference levels for a particular disease state, phenotype, or lack thereof in a certain age or gender group). Such reference levels may also be tailored to specific techniques that are used to measure levels of biomarkers in biological samples (e.g., PCR, microarray analysis, mass spectrometry (e.g., LC-MS, GC-MS), tandem mass spectrometry, NMR, biochemical or enzymatic assays, etc.), where the levels of biomarkers may differ based on the specific technique that is used.

A “similarity value” is a number that represents the degree of similarity between two things being compared. For example, a similarity value may be a number that indicates the overall similarity between a patient's biomarker profile using specific phenotype-related biomarkers and reference value ranges for the biomarkers in one or more control samples or a reference profile (e.g., the similarity to a “insulin resistance” biomarker profile or a “type 2 diabetes” biomarker profile). The similarity value may be expressed as a similarity metric, such as a correlation coefficient, or may simply be expressed as the expression level difference, or the aggregate of the expression level differences, between levels of biomarkers in a patient sample and a control sample or reference expression profile.

The terms “quantity”, “amount”, and “level” are used interchangeably herein and may refer to an absolute quantification of a molecule or an analyte in a sample, or to a relative quantification of a molecule or analyte in a sample, i.e., relative to another value such as relative to a reference value as taught herein, or to a range of values for the biomarker. These values or ranges can be obtained from a single patient or from a group of patients.

Biological sample. The term “sample” with respect to an individual encompasses blood and other liquid samples of biological origin, solid tissue samples such as a biopsy specimen or tissue cultures or cells derived or isolated therefrom and the progeny thereof. The definition also includes samples that have been manipulated in any way after their procurement, such as by treatment with reagents; washed; or enrichment for certain cell populations, such as PBMCs, fibroblasts, keratinocytes, epithelial cells, endothelial progenitor cells, or other cells that can be used to generate IPSC-ECs. The definition also includes samples that have been enriched for particular types of molecules, e.g., nucleic acids, polypeptides, etc.

“Endothelial progenitor cells” include circulating cells that express a variety of cell surface markers similar to those expressed by vascular endothelial cells (e.g., CD34, CD133, and/or KDR), and may adhere to endothelium, particularly at sites of hypoxia/ischemia, and participate in new vessel formation. Endothelial progenitor cells produce progeny displaying clonal proliferative potential and differentiation restricted to the endothelial lineage. Such endothelial progenitor cells should have an ability to form lumenized capillary-like tubes in vitro, an ability to form human blood vessels, and an ability to undergo remodeling to form the intima of arterial, venous, and capillary structures.

The term “biological sample” encompasses a clinical sample. The types of “biological samples” include, but are not limited to: tissue obtained by surgical resection, tissue obtained by biopsy, cells in culture, cell supernatants, cell lysates, tissue samples, organs, bone marrow, blood, plasma, serum, fine needle aspirate, lymph node aspirate, cystic aspirate, a paracentesis sample, a thoracentesis sample, and the like. A “biological sample” can include cells (e.g., target cells, normal cells, blood cells, tissue cells etc.) can be suspected of comprising such cells, or can be devoid of cells. A biological sample can include biological fluids derived from cells (e.g., an insulin resistant cell, etc.), e.g., a sample comprising polynucleotides and/or polypeptides that is obtained from such cells (e.g., a cell lysate or other cell extract comprising polynucleotides and/or polypeptides). A biological sample comprising an inflicted cell from a patient can also include non-inflicted cells. In some embodiments the biological sample is blood or a derivative thereof, e.g. plasma, serum, etc.

Obtaining and assaying a sample. The term “assaying” is used herein to include the physical steps of manipulating a biological sample to generate data related to the sample. As will be readily understood by one of ordinary skill in the art, a biological sample must be “obtained” prior to assaying the sample. Thus, the term “assaying” implies that the sample has been obtained. The terms “obtained” or “obtaining” as used herein encompass the act of receiving an extracted or isolated biological sample. For example, a testing facility can “obtain” a biological sample in the mail (or via delivery, etc.) prior to assaying the sample. In some such cases, the biological sample was “extracted” or “isolated” from an individual by another party prior to mailing (i.e., delivery, transfer, etc.), and then “obtained” by the testing facility upon arrival of the sample. Thus, a testing facility can obtain the sample and then assay the sample, thereby producing data related to the sample.

The terms “obtained” or “obtaining” as used herein can also include the physical extraction or isolation of a biological sample from a subject. Accordingly, a biological sample can be isolated from a subject (and thus “obtained”) by the same person or same entity that subsequently assays the sample. When a biological sample is “extracted” or “isolated” from a first party or entity and then transferred (e.g., delivered, mailed, etc.) to a second party, the sample was “obtained” by the first party (and also “isolated” by the first party), and then subsequently “obtained” (but not “isolated”) by the second party. Accordingly, in some embodiments, the step of obtaining does not comprise the step of isolating a biological sample.

In some embodiments, the step of obtaining comprises the step of isolating a biological sample (e.g., a pre-treatment biological sample, a post-treatment biological sample, etc.). Methods and protocols for isolating various biological samples (e.g., a blood sample, a serum sample, a plasma sample, a biopsy sample, an aspirate, etc.) will be known to one of ordinary skill in the art and any convenient method may be used to isolate a biological sample.

It will be understood by one of ordinary skill in the art that in some cases, it is convenient to wait until multiple samples (e.g., a pre-treatment biological sample and a post-treatment biological sample) have been obtained prior to assaying the samples. Accordingly, in some cases an isolated biological sample (e.g., a pre-treatment biological sample, a post-treatment biological sample, etc.) is stored until all appropriate samples have been obtained. One of ordinary skill in the art will understand how to appropriately store a variety of different types of biological samples and any convenient method of storage may be used (e.g., refrigeration) that is appropriate for the particular biological sample. In some embodiments, a pre-treatment biological sample is assayed prior to obtaining a post-treatment biological sample. In some cases, a pre-treatment biological sample and a post-treatment biological sample are assayed in parallel. In some cases, multiple different post-treatment biological samples and/or a pre-treatment biological sample are assayed in parallel. In some cases, biological samples are processed immediately or as soon as possible after they are obtained.

In some embodiments, the concentration (i.e., “level”), or expression level of a gene product, which may be an RNA, a protein, etc., (which will be referenced herein as a biomarker), in a biological sample is measured (i.e., “determined”). By “expression level” (or “level”) it is meant the level of gene product (e.g. the absolute and/or normalized value determined for the RNA expression level of a biomarker or for the expression level of the encoded polypeptide, or the concentration of the protein in a biological sample). The term “gene product” or “expression product” are used herein to refer to the RNA transcription products (RNA transcripts, e.g. mRNA, an unspliced RNA, a splice variant mRNA, and/or a fragmented RNA) of the gene, including mRNA, and the polypeptide translation products of such RNA transcripts. A gene product can be, for example, an unspliced RNA, an mRNA, a splice variant mRNA, a microRNA, a fragmented RNA, a polypeptide, a post-translationally modified polypeptide, a splice variant polypeptide, etc.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assaying may be relative or absolute. For example, “assaying” can be determining whether the expression level is less than or “greater than or equal to” a particular threshold, (the threshold can be pre-determined or can be determined by assaying a control sample). On the other hand, “assaying to determine the expression level” can mean determining a quantitative value (using any convenient metric) that represents the level of expression (i.e., expression level, e.g., the amount of protein and/or RNA, e.g., mRNA) of a particular biomarker. The level of expression can be expressed in arbitrary units associated with a particular assay (e.g., fluorescence units, e.g., mean fluorescence intensity (MFI)), or can be expressed as an absolute value with defined units (e.g., number of mRNA transcripts, number of protein molecules, concentration of protein, etc.). Additionally, the level of expression of a biomarker can be compared to the expression level of one or more additional genes (e.g., nucleic acids and/or their encoded proteins) to derive a normalized value that represents a normalized expression level. The specific metric (or units) chosen is not crucial as long as the same units are used (or conversion to the same units is performed) when evaluating multiple biological samples from the same individual (e.g., biological samples taken at different points in time from the same individual). This is because the units cancel when calculating a fold-change (i.e., determining a ratio) in the expression level from one biological sample to the next (e.g., biological samples taken at different points in time from the same individual).

For measuring RNA levels, the amount or level of an RNA in the sample is determined, e.g., the level of an mRNA. In some instances, the expression level of one or more additional RNAs may also be measured, and the level of biomarker expression compared to the level of the one or more additional RNAs to provide a normalized value for the biomarker expression level. Any convenient protocol for evaluating RNA levels may be employed wherein the level of one or more RNAs in the assayed sample is determined.

A number of exemplary methods for measuring RNA (e.g., mRNA) expression levels (e.g., expression level of a nucleic acid biomarker) in a sample are known by one of ordinary skill in the art, and any convenient method can be used. Exemplary methods include, but are not limited to: hybridization-based methods (e.g., Northern blotting, array hybridization (e.g., microarray); in situ hybridization; in situ hybridization followed by FACS; and the like)(Parker & Barnes, Methods in Molecular Biology 106:247-283 (1999)); RNase protection assays (Hod, Biotechniques 13:852-854 (1992)); PCR-based methods (e.g., reverse transcription PCR (RT-PCR), quantitative RT-PCR (qRT-PCR), real-time RT-PCR, etc.)(Weis et al., Trends in Genetics 8:263-264 (1992)); nucleic acid sequencing methods (e.g., Sanger sequencing, Next Generation sequencing (i.e., massive parallel high throughput sequencing, e.g., Illumina's reversible terminator method, Roche's pyrosequencing method (454), Life Technologies' sequencing by ligation (the SOLiD platform), Life Technologies' Ion Torrent platform, single molecule sequencing, etc.); and the like.

In some embodiments, the IPSC-ECs can be assayed directly. In some embodiments, nucleic acids of the IPSC-ECs are amplified (e.g., by PCR) prior to assaying. As such, techniques such as PCR (Polymerase Chain Reaction), RT-PCR (reverse transcriptase PCR), qRT-PCR (quantitative RT-PCR, real time RT-PCR), etc. can be used prior to the hybridization methods and/or the sequencing methods discussed above.

For measuring mRNA levels, the starting material is typically total RNA or poly A+ RNA isolated from IPSC-ECs derived from somatic cells of a biological sample (e.g., suspension of cells from a peripheral blood sample, a bone marrow sample, etc., or from a homogenized tissue, e.g. a homogenized biopsy sample, an aspirate, a homogenized paraffin- or OCT-embedded sample, etc.). General methods for mRNA extraction are well known in the art and are disclosed in standard textbooks of molecular biology, including Ausubel et al., Current Protocols of Molecular Biology, John Wiley and Sons (1997). RNA isolation can also be performed using a purification kit, buffer set and protease from commercial manufacturers, according to the manufacturer's instructions. For example, RNA from cell suspensions can be isolated using Qiagen RNeasy mini-columns, and RNA from cell suspensions or homogenized tissue samples can be isolated using the TRIzol reagent-based kits (Invitrogen), MasterPure Complete DNA and RNA Purification Kit (EPICENTRE™, Madison, Wis.), Paraffin Block RNA Isolation Kit (Ambion, Inc.) or RNA Stat-60 kit (Tel-Test).

A variety of different manners of measuring mRNA levels are known in the art, e.g. as employed in the field of differential gene expression analysis. One representative and convenient type of protocol for measuring mRNA levels is array-based gene expression profiling. Such protocols are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively.

Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed. The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

The resultant pattern of hybridized nucleic acid provides information regarding expression for each of the genes that have been probed, where the expression information is in terms of whether or not the gene is expressed and, typically, at what level, where the expression data, i.e., expression profile (e.g., in the form of a transcriptosome), may be both qualitative and quantitative.

Alternatively, non-array based methods for quantitating the level of one or more nucleic acids in a sample may be employed. These include those based on amplification protocols, e.g., Polymerase Chain Reaction (PCR)-based assays, including quantitative PCR, reverse-transcription PCR (RT-PCR), real-time PCR, and the like, e.g. TaqMan® RT-PCR, MassARRAY® System, BeadArray® technology, and Luminex technology; and those that rely upon hybridization of probes to filters, e.g. Northern blotting and in situ hybridization.

Examples of some of the nucleic acid sequencing methods listed above are described in the following references: Margulies et al (Nature 2005 437: 376-80); Ronaghi et al (Analytical Biochemistry 1996 242: 84-9); Shendure (Science 2005 309: 1728); Imelfort et al (Brief Bioinform. 2009 10:609-18); Fox et al (Methods Mol Biol. 2009; 553:79-108); Appleby et al (Methods Mol Biol. 2009; 513:19-39) and Morozova (Genomics. 2008 92:255-64), which are incorporated by reference for the general descriptions of the methods and the particular steps of the methods, including all starting products, reagents, and final products for each of the steps.

For measuring protein levels, the amount or level of a protein in IPSC-ECs derived from somatic cells in the biological sample is determined. In some cases, the polypeptide comprises a post-translational modification (e.g., phosphorylation) associated with regulation of activity of the protein such as by a signaling cascade, wherein the modified protein is the biomarker (e.g., phosphorylated Akt), and the amount of the modified protein is therefore measured. In some embodiments, an extracellular protein level is measured. For example, in some cases, the protein (i.e., polypeptide) being measured is a secreted protein (e.g., a cytokine or chemokine) and the concentration can therefore be measured in extracellular fluid. In some embodiments, concentration is a relative value measured by comparing the level of one protein relative to another protein. In other embodiments the concentration is an absolute measurement of weight/volume or weight/weight.

In some cases, the cells are removed from the biological sample (e.g., via centrifugation, via adhering cells to a dish or to plastic, etc.) prior to measuring the concentration. In some cases, the intracellular protein level is measured by lysing the removed cells of the biological sample to measure the level of protein in the cellular contents. In some cases, both the extracellular and intracellular levels of protein are measured by separating the cellular and fluid portions of the biological sample (e.g., via centrifugation), measuring the extracellular level of the protein by measuring the level of protein in the fluid portion of the biological sample, and measuring the intracellular level of protein by measuring the level of protein in the cellular portion of the biological sample (e.g., after lysing the cells). In some cases, the total level of protein (i.e., combined extracellular and intracellular protein) is measured by lysing the cells of the biological sample to include the intracellular contents as part of the sample.

In some instances, the concentration of one or more additional proteins may also be measured, and biomarker concentration compared to the level of the one or more additional proteins to provide a normalized value for the biomarker concentration. Any convenient protocol for evaluating protein levels may be employed wherein the level of one or more proteins in the assayed sample is determined.

While a variety of different manners of assaying for protein levels are known to one of ordinary skill in the art and any convenient method may be used, one representative and convenient type of protocol for assaying protein levels is ELISA, an antibody-based method. In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Following incubation, the antisera-contacted surface is washed so as to remove non-immunocomplexed material. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H2O2, in the case of a peroxidase label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody. The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatographic column or filter with a wash solution or solvent.

Alternatively, non-ELISA based-methods for measuring the levels of one or more proteins in a sample may be employed. Representative exemplary methods include but are not limited to antibody-based methods (e.g., Western blotting, proteomic arrays, xMAP microsphere technology (e.g., Luminex technology), immunohistochemistry, flow cytometry, and the like) as well as non-antibody-based methods (e.g., mass spectrometry or tandem mass spectrometry).

“Diagnosis” as used herein generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. By “pluripotency” and pluripotent stem cells it is meant that such cells have the ability to differentiate into all types of cells in an organism.

Additional Terms.

The term “induced pluripotent stem cell” encompasses pluripotent cells, that, like embryonic stem (ES) cells, can be cultured over a long period of time while maintaining the ability to differentiate into all types of cells in an organism, but that, unlike ES cells (which are derived from the inner cell mass of blastocysts), are derived from differentiated somatic cells, that is, cells that had a narrower, more defined potential and that in the absence of experimental manipulation could not give rise to all types of cells in the organism. PS cells have an hESC-like morphology, growing as flat colonies with large nucleo-cytoplasmic ratios, defined borders and prominent nuclei. In addition, PS cells express one or more key pluripotency markers known by one of ordinary skill in the art, including but not limited to alkaline phosphatase, SSEA3, SSEA4, Sox2, Oct3/4, Nanog, TRA160, TRA181, TDGF 1, Dnmt3b, FoxD3, GDF3, Cyp26a1, TERT, and zfp42. In addition, the iPS cells are capable of forming teratomas. In addition, they are capable of forming or contributing to ectoderm, mesoderm, or endoderm tissues in a living organism.

As used herein, “reprogramming factors” refers to one or more, i.e. a cocktail, of biologically active factors that act on a cell to alter transcription, thereby reprogramming a cell to multipotency or to pluripotency. Reprogramming factors may be provided to the cells, e.g. somatic cells from an individual with a family history or genetic make-up of interest for insulin resistance and/or type 2 diabetes such as peripheral blood mononuclear cells (PBMCs), fibroblasts, keratinocytes, epithelial cells, or endothelial progenitor cells, etc.; individually or as a single composition, that is, as a premixed composition, of reprogramming factors. The factors may be provided at the same molar ratio or at different molar ratios. The factors may be provided once or multiple times in the course of culturing the cells of the subject invention. In some embodiments the reprogramming factor is a transcription factor, including without limitation, Oct3/4; Sox2; Klf4; c-Myc; Nanog; and Lin-28.

Somatic cells, including, without limitation, peripheral blood mononuclear cells, fibroblasts, keratinocytes, epithelial cells, endothelial progenitor cells, adipose derived stem cells, etc., are contacted with reprogramming factors, as defined above, in a combination and quantity sufficient to reprogram the cell to pluripotency. Reprogramming factors may be provided to the somatic cells individually or as a single composition, that is, as a premixed composition, of reprogramming factors. In some embodiments the reprogramming factors are provided as a plurality of coding sequences on a vector.

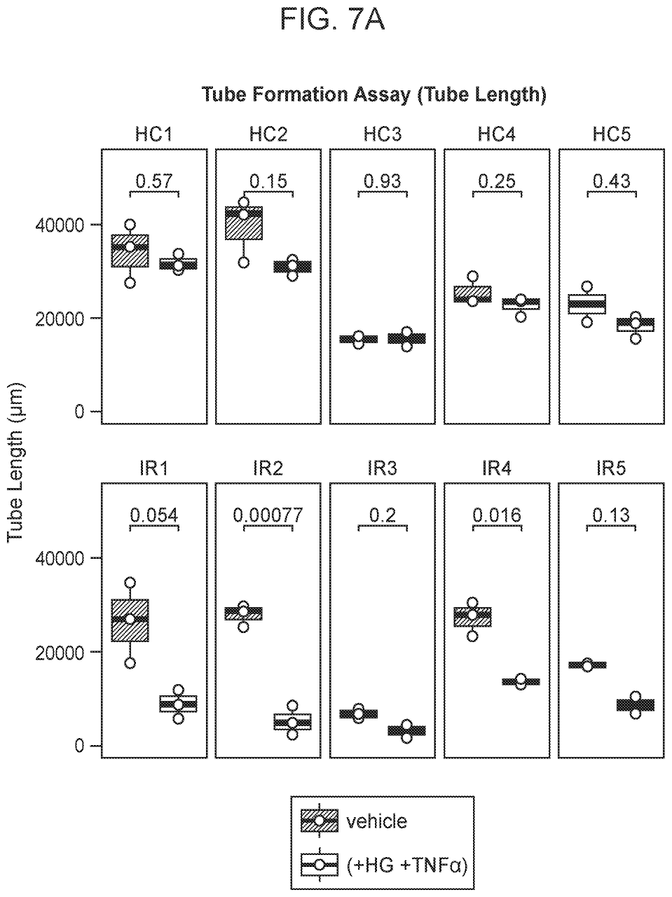

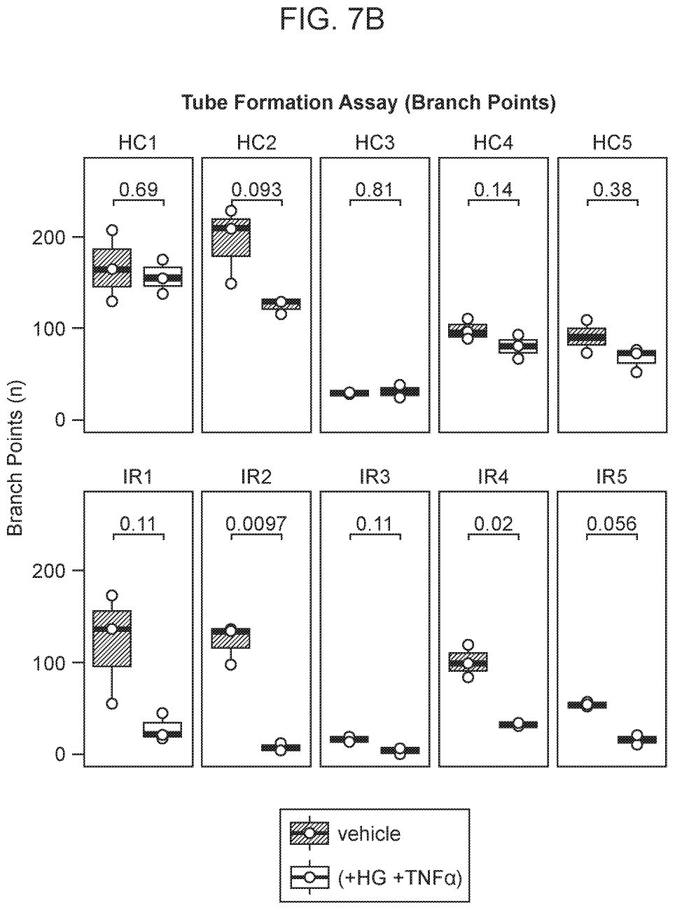

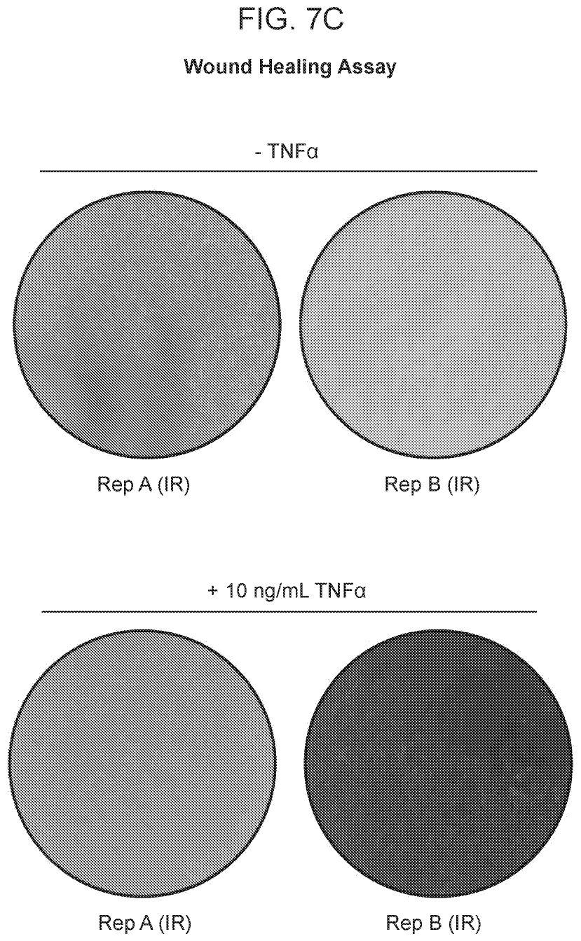

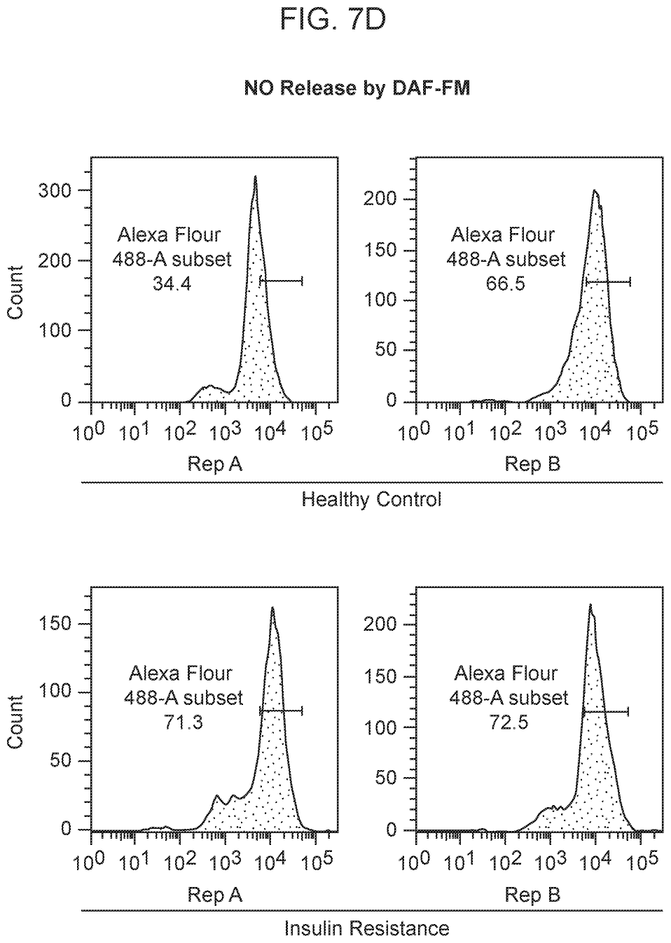

Differentiation of PS cells into endothelial cells (iPSC-ECs) may be promoted by including various growth factors and other differentiation agents in the culture, such as vascular endothelial growth factor (VEGF), basic fibroblast growth factor (bFGF), and bone morphogenetic protein 4 (BMP4). See, e.g., Examples and FIG. 7A. Further examples of such protocols are described by Yoder (2015) Curr Opin Hematol. 22(3):252-7, Lin et al. (2017) Arterioscler Thromb Vasc Biol. 37(11):2014-2025, and Harding et al (2017) Stem Cells 35(4):909-919; herein incorporated by reference. However, any suitable method of inducing endothelial cell differentiation may be used.

The iPSC-ECs are harvested at an appropriate stage of development, which may be determined based on the expression of markers and phenotypic characteristics of the desired endothelial cell type e.g. at from about 1 to 4 weeks. Cultures may be empirically tested by staining for the presence of the markers of interest, by morphological determination, etc. The cells are optionally enriched before or after the positive selection step by drug selection, panning, density gradient centrifugation, etc. In another embodiment, a negative selection is performed, where the selection is based on expression of one or more of the markers found on PBMCs, fibroblasts, epithelial cells, endothelial progenitor cells, and the like. Selection may utilize panning methods, magnetic particle selection, particle sorter selection, and the like.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with cancer, those with an infection, etc.) as well as those in which prevention is desired (e.g., those with increased susceptibility to cancer, those with an increased likelihood of infection, those suspected of having cancer, those suspected of harboring an infection, etc.).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

The term “about,” particularly in reference to a given quantity, is meant to encompass deviations of plus or minus five percent.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

The terms “diabetes” and “diabetic” refer to a progressive disease of carbohydrate metabolism involving inadequate production or utilization of insulin, frequently characterized by hyperglycemia and glycosuria. The terms “pre-diabetes” and “pre-diabetic” refer to a state wherein a subject does not have the characteristics, symptoms and the like typically observed in diabetes, but does have characteristics, symptoms and the like that, if left untreated, may progress to diabetes. The presence of these conditions may be determined using, for example, either the fasting plasma glucose (FPG) test or the oral glucose tolerance test (OGTT). Both usually require a subject to fast for at least 8 hours prior to initiating the test. In the FPG test, a subject's blood glucose is measured after the conclusion of the fasting; generally, the subject fasts overnight and the blood glucose is measured in the morning before the subject eats. A healthy subject would generally have a FPG concentration between about 90 and about 100 mg/dl, a subject with “pre-diabetes” would generally have a FPG concentration between about 100 and about 125 mg/dl, and a subject with “diabetes” would generally have a FPG level above about 126 mg/dl. In the OGTT, a subject's blood glucose is measured after fasting and again two hours after drinking a glucose-rich beverage. Two hours after consumption of the glucose-rich beverage, a healthy subject generally has a blood glucose concentration below about 140 mg/dl, a pre-diabetic subject generally has a blood glucose concentration about 140 to about 199 mg/dl, and a diabetic subject generally has a blood glucose concentration about 200 mg/dl or above. The term “insulin resistance” as used herein refers to a condition where a normal amount of insulin is unable to produce a normal physiological or molecular response. In some cases, a hyper-physiological amount of insulin, either endogenously produced or exogenously administered, is able to overcome the insulin resistance, in whole or in part, and produce a biologic response.

The term “metabolic syndrome” refers to an associated cluster of traits that includes, but is not limited to, hyperinsulinemia, abnormal glucose tolerance, obesity, redistribution of fat to the abdominal or upper body compartment, hypertension, dysfibrinolysis, and dyslipidemia characterized by high triglycerides, low high density lipoprotein (HDL)-cholesterol, and high small dense low density lipoprotein (LDL) particles. Subjects having metabolic syndrome are at risk for development of type 2 diabetes and/or other disorders (e.g., atherosclerosis).

The phrase “glucose metabolism disorder” encompasses any disorder characterized by a clinical symptom or a combination of clinical symptoms that is associated with an elevated level of glucose and/or an elevated level of insulin in a subject relative to a healthy individual. Elevated levels of glucose and/or insulin may be manifested in the following diseases, disorders and conditions: hyperglycemia, type 2 diabetes, gestational diabetes, type 1 diabetes, insulin resistance, impaired glucose tolerance, hyperinsulinemia, impaired glucose metabolism, pre-diabetes, other metabolic disorders (such as metabolic syndrome, which is also referred to as syndrome X), and obesity, among others.

The term “hyperglycemia”, as used herein, refers to a condition in which an elevated amount of glucose circulates in the blood plasma of a subject relative to a healthy individual. Hyperglycemia can be diagnosed using methods known in the art, including measurement of fasting blood glucose levels as described herein.

The term “hyperinsulinemia”, as used herein, refers to a condition in which there are elevated levels of circulating insulin when, concomitantly, blood glucose levels are either elevated or normal. Hyperinsulinemia can be caused by insulin resistance which is associated with dyslipidemia, such as high triglycerides, high cholesterol, high low-density lipoprotein (LDL) and low high-density lipoprotein (HDL); high uric acids levels; polycystic ovary syndrome; type 2 diabetes and obesity. Hyperinsulinemia can be diagnosed as having a plasma insulin level higher than about 2 U/mL.

As used herein, the phrase “body weight disorder” refers to conditions associated with excessive body weight and/or enhanced appetite. Various parameters are used to determine whether a subject is overweight compared to a reference healthy individual, including the subject's age, height, sex and health status. For example, a subject may be considered overweight or obese by assessment of the subject's Body Mass Index (BMI), which is calculated by dividing a subject's weight in kilograms by the subject's height in meters squared. An adult having a BMI in the range of about 18.5 to about.24.9 kg/m2 is considered to have a normal weight; an adult having a BMI between about 25 and about 29.9 kg/m² may be considered overweight (pre-obese); and an adult having a BMI of about 30 kg/m² or higher may be considered obese. Enhanced appetite frequently contributes to excessive body weight. There are several conditions associated with enhanced appetite, including, for example, night eating syndrome, which is characterized by morning anorexia and evening polyphagia often associated with insomnia, but which may be related to injury to the hypothalamus.

A “therapeutically effective dose” or “therapeutic dose” is an amount sufficient to effect desired clinical results (i.e., achieve therapeutic efficacy). A therapeutically effective dose can be administered in one or more administrations.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer. Both full-length proteins and fragments thereof are encompassed by the definition. The terms also include postexpression modifications of the polypeptide, for example, phosphorylation, glycosylation, acetylation, hydroxylation, oxidation, and the like.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), and any other type of polynucleotide which is an N- or C-glycoside of a purine or pyrimidine base. There is no intended distinction in length between the terms “polynucleotide,” “oligonucleotide,” “nucleic acid” and “nucleic acid molecule,” and these terms are used interchangeably.

By “isolated” is meant, when referring to a protein, polypeptide, or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

“Providing an analysis” is used herein to refer to the delivery of an oral or written analysis (i.e., a document, a report, etc.). A written analysis can be a printed or electronic document. A suitable analysis (e.g., an oral or written report) provides any or all of the following information: identifying information of the subject (name, age, etc.), a description of what type of biological sample(s) was used and/or how it was used, the technique used to assay the sample, the results of the assay (e.g., the level of the biomarker as measured in the pre-treatment and post-treatment assay and/or the fold-change of a biomarker level in a post-treatment assay compared to a pre-treatment assay), the assessment as to whether the individual is determined to be responsive or not responsive to the pre-diabetic treatment, the assessment as to whether the individual is determined to be at risk of developing insulin resistance and/or disease progression to type 2 diabetes, a recommendation to continue or alter therapy, a recommended strategy for additional therapy, etc. The report can be in any format including, but not limited to printed information on a suitable medium or substrate (e.g., paper); or electronic format. If in electronic format, the report can be in any computer readable medium, e.g., diskette, compact disk (CD), flash drive, and the like, on which the information has been recorded. In addition, the report may be present as a website address which may be used via the internet to access the information at a remote site.

### Biomarkers and Diagnostic Methods

Biomarkers that can be used in the practice of the subject methods include, without limitation, phosphorylated Akt, reactive oxygen species (ROS), NO, SIRT1, eNOS, CDH13, and IRS1. Aberrant changes in the levels of these biomarkers are associated with development of insulin resistance. Accordingly, monitoring the levels of these biomarkers is useful for determining the risk of an individual developing insulin resistance.

Genes, loci and variants associated with diabetes, cardiovascular disease and insulin resistance in genome wide association studies (GWAS) and exosomal microRNA from iPSC-EC are potential biomarkers for insulin resistance (Table 4).

The diagnostic methods described herein can be used, for example, for evaluating subjects who have not yet developed the pathological changes that cause clinical signs and symptoms of pre-diabetes or diabetes and consequent vascular disease. In particular, the methods described herein are useful for monitoring an individual who has known risk factors for developing insulin resistance and/or type 2 diabetes, such as, but not limited to, obesity, inadequate exercise (i.e., sedentary lifestyle), a genetic predisposition for developing diabetes, a family history indicating a risk of developing diabetes (e.g., first degree relatives with diabetes), a disease associated with insulin resistance (e.g., polycystic ovary syndrome, hepatitis C), high blood pressure, high cholesterol, age of 45 or older, history of gestational diabetes, vitamin D deficiency, treatment with HIV drugs having protease inhibitors, or growth hormone replacement therapy. The method can be performed, for example, on samples from children with a strong family history indicating a risk of developing insulin resistance or type 2 diabetes, wherein if a child is identified as being at risk of developing insulin resistance, lifestyle changes (e.g., diet modification, weight control, adequate exercise, etc., as discussed further below) can be recommended early in life to minimize the risk and potentially avoid the pathological physiological changes leading to insulin resistance or type 2 diabetes.

The diagnostic methods for determining whether an individual is at risk of developing insulin resistance typically comprise the steps of a) obtaining a biological sample comprising somatic cells from an individual; b) generating induced pluripotent stem cells (IPSCs) from the somatic cells; c) differentiating the IPSCs into endothelial cells (IPSC-ECs) or vascular smooth muscle cells (IPSC-VSMCs); and d) measuring one or more of the biomarkers described herein in the IPSC-ECs or IPSC-VSMCs derived from the somatic cells of the individual. The levels of the biomarkers in the IPSC-ECs or IPSC-VSMCs derived from the somatic cells of the individual are compared with respective reference value ranges for the biomarkers in control IPSC-ECs or IPSC-VSMCs (i.e., derived from somatic cells of a normal or healthy subject (e.g. an individual known to not have insulin resistance or diabetes).

When analyzing the levels of biomarkers in IPSC-ECs or IPSC-VSMCs derived from a subject, the reference value ranges used for comparison can represent the levels of one or more biomarkers in IPSC-ECs or IPSC-VSMCs derived from one or more subjects without insulin resistance (i.e., normal or healthy control), wherein detection of reduced phosphorylation of Akt, increased levels of ROS, attenuated levels of NO production, decreased levels of expression of SIRT1 and IRS1, and increased levels of expression of eNOS and CDH13 in the IPSC-ECs or IPSC-VSMCs derived from the subject compared to reference values ranges for levels of the biomarkers in control IPSC-ECs or IPSC-VSMCs indicate the subject is at risk of developing insulin resistance. Alternatively, the reference values can represent the levels of one or more biomarkers in IPSC-ECs or IPSC-VSMCs derived from one or more subjects with insulin resistance or type 2 diabetes, wherein similarity to the reference value ranges indicates the stage of disease progression (e.g., high risk of insulin resistance, severity of insulin resistance, or type 2 diabetes).

A sample comprising somatic cells is obtained from the subject. The somatic cells may include, without limitation, peripheral blood mononuclear cells, fibroblasts, keratinocytes, epithelial cells, endothelial progenitor cells, adipose derived stem cells, and other cell types capable of generating patient-derived IPSC-ECs or IPSC-VSMCs, The biological sample comprising somatic cells is typically whole blood, buffy coat, peripheral blood mononucleated cells (PBMCS), skin, fat, or a biopsy, but can be any sample from bodily fluids, tissue or cells that contain suitable somatic cells. A “control” sample, as used herein, refers to a biological sample comprising somatic cells, such as a bodily fluid, tissue, or cells that are not diseased. That is, a control sample is obtained from a normal or healthy subject (e.g. an individual known to not have insulin resistance). A biological sample can be obtained from a subject by conventional techniques. For example, blood can be obtained by venipuncture, and solid tissue samples can be obtained by surgical techniques according to methods well known in the art.

The IPSC-ECs or IPSC-VSMCs can be generated by reprogramming the somatic cells obtained from the subject into pluripotent stem cells followed by redifferentiation into endothelial cells or smooth muscle cells, respectively. Somatic cells can be induced into forming pluripotent stem cells, for example, by treating them with reprograming factors such as Yamanaka factors, including but not limited to, OCT3, OCT4, SOX2, KLF4, c-MYC, NANOG, and LIN28 (see, e.g., Takahashi et al. (2007) Cell. 131(5):861-872; herein incorporated by reference in its entirety). Somatic cells are contacted with reprogramming factors in a combination and quantity sufficient to reprogram the cells to pluripotency. Reprogramming factors may be provided to the somatic cells individually or as a single composition, that is, as a premixed composition, of reprogramming factors. In some embodiments the reprogramming factors are provided as a plurality of coding sequences on a vector.

Methods for “introducing a cell reprogramming factor into somatic cells are not limited in particular, and known procedures can be selected and used as appropriate. For example, when a cell reprogramming factor as described above is introduced into somatic cells of the above-mentioned type in the form of proteins, such methods include ones using protein introducing reagents, fusion proteins with protein transfer domains (PTDs), electroporation, and microinjection. When a cell reprogramming factor as described above is introduced into somatic cells of the above-mentioned type in the form of nucleic acids encoding the cell reprogramming factor, a nucleic acid(s), such as cDNA(s), encoding the cell reprogramming factor can be inserted in an appropriate expression vector comprising a promoter that functions in somatic cells, which then can be introduced into somatic cells by procedures such as infection, lipofection, liposomes, electroporation, calcium phosphate coprecipitation, DEAE-dextran, microinjection, and electroporation. Examples of an “expression vector” include viral vectors, such as lentiviruses, retroviruses, adenoviruses, adeno-associated viruses, and herpes viruses; and expression plasmids for animal cells. For example, retroviral or Sendai virus (SeV) vectors are commonly used to introduce a nucleic acid(s) encoding a cell reprogramming factor as described above into somatic cells.

After in vitro expansion, the IPSCs derived from the somatic cells can be redifferentiated into endothelial cells by culturing the IPSCs in the presence of various growth factors and other differentiation agents such as vascular endothelial growth factor (VEGF), basic fibroblast growth factor (bFGF), and bone morphogenetic protein 4 (BMP4). For redifferentiation protocols, see, e.g., Example 1 and FIG. 7A. Further examples of such protocols are described by Yoder (2015) Curr Opin Hematol. 22(3):252-7, Lin et al. (2017) Arterioscler Thromb Vasc Biol. 37(11):2014-2025, and Harding et al (2017) Stem Cells 35(4):909-919; herein incorporated by reference. However, any suitable method of inducing endothelial cell differentiation may be used.

The iPSC-ECs are harvested at an appropriate stage of development, which may be determined based on the expression of markers and phenotypic characteristics of the desired endothelial cell type e.g. at from about 1 to 4 weeks. Cultures may be empirically tested by staining for the presence of one or more endothelial cell markers of interest (e.g., CD13, CD29, CD31, CD34, CD36, CD39, CD44, CD47, CD54, CD61, CD62, CD80, CD86, CD93, CD102, CD105, CD106, CD112, CD117, CD121, CD141, CD142, CD143, CD144, CD146, CD147, CD151, CD160, CD201, and CD309), by morphological determination, etc. The cells are optionally enriched before or after a positive selection step by drug selection, panning, density gradient centrifugation, etc. In another embodiment, a negative selection is performed, where the selection is based on expression of one or more of the markers found on the somatic cells (e.g., PBMCs, fibroblasts, keratinocytes, epithelial cells, endothelial progenitor cells, and the like). Selection may utilize panning methods, magnetic particle selection, particle sorter selection, and the like.

The methods described herein may be used to determine if a patient should be treated for insulin resistance. For example, a patient is selected for a pre-diabetic treatment if the subject is determined to be at risk of developing insulin resistance. The pre-diabetic treatment may comprise, for example, without limitation, administering a therapeutically effective amount of metformin to the subject, modifying diet of the subject to reduce carbohydrate intake, reducing weight of the subject, increasing amount of exercise of the subject, bariatric surgery, administering medication to treat high blood pressure in the subject, administering medication to treat high cholesterol in the subject, ceasing smoking of the subject, or a combination thereof. In some embodiments, the treatment further comprises administering to the subject a sulfonylurea (e.g., acetohexamide, carbutamide, chlorpropamide, glycyclamide (tolhexamide), metahexamide, tolazamide, tolbutamide, glibenclamide (glyburide), glibornuride, gliclazide, glipizide, gliquidone, glisoxepide, glyclopyramide, and glimepiride), a thiazolidinedione (e.g., pioglitazone, rosiglitazone, lobeglitazone), a dipeptidyl peptidase-4 inhibitor (e.g., sitagliptin, vildagliptin, saxagliptin, linagliptin, gemigliptin, anagliptin, teneligliptin, alogliptin, trelagliptin, omarigliptin, evogliptin, gosogliptin, and dutogliptin), a sodium-glucose transport protein 2 (SGLT2) inhibitor (e.g., canagliflozin, dapagliflozin, empagliflozin, ertugliflozin, ipragliflozin, luseogliflozin, remogliflozin etabonate, sotagliflozin, and tofogliflozin), a glucagon-like peptide-1 analog (e.g., exenatide, liraglutide, lixisenatide, albiglutide, dulaglutide, and semaglutide), an angiotensin-converting enzyme inhibitor (e.g., benazepril, zofenopril, perindopril, trandolapril, captopril, enalapril, lisinopril, and ramipril), or a combination thereof.

In some embodiments, the methods described herein are used for monitoring progression of prediabetes in a subject. For example, a first biological sample comprising somatic cells can be obtained from the subject at a first time point and a second biological sample comprising somatic cells can be obtained from the subject later at a second time point. Induced pluripotent stem cells (IPSCs) are generated from the somatic cells in the first sample and the second sample and differentiated into endothelial cells (IPSC-ECs) followed by measuring one or more biomarkers in the IPSC-ECs derived from the subject. In some cases, combinations of biomarkers are used. In some such cases, the levels of all measured biomarkers must change (as described above) in order for the determination to be made regarding the risk of the subject developing insulin resistance and/or progression of prediabetes in the subject. In some embodiments, phosphorylation of Akt, levels of reactive oxygen species (ROS), levels of NO, and levels of expression of SIRT1, eNOS, CDH13, and IRS1 are used for evaluating progression of prediabetes in the subject, wherein detection of reduced phosphorylation of Akt, increased levels of ROS, decreased levels of NO, decreased levels of expression of SIRT1 and IRS1, and increased levels of expression of eNOS and CDH13 in the IPSC-ECs derived from the somatic cells of the second sample compared to the IPSC-ECs derived from the somatic cells of the first sample indicates that the risk of the subject developing insulin resistance is increasing, and detection of increased phosphorylation of Akt, decreased levels of ROS, increased levels of NO, increased levels of expression of SIRT1 and IRS1, and decreased levels of expression of eNOS and CDH13 in the IPSC-ECs derived from the somatic cells of the second sample compared to the IPSC-ECs derived from the somatic cells of the first sample indicates that the risk of the subject developing insulin resistance is decreasing.

The subject methods may also be used for assaying pre-treatment and post-treatment biological samples isolated from an individual to determine whether the individual is responsive or not responsive to a pre-diabetic treatment. For example, a first biological sample comprising somatic cells can be obtained from the subject before the subject undergoes the therapy, and a second biological sample comprising somatic cells can be obtained from the subject after the subject undergoes the therapy. Induced pluripotent stem cells (IPSCs) are generated from the somatic cells in the first sample and the second sample and differentiated into endothelial cells (IPSC-ECs) followed by measuring one or more biomarkers in the IPSC-ECs derived from the subject. To simulate the hyperglycemic and pro-inflammatory environment found in patients, IPSC-ECs may be treated with a mimicking cocktail, for example, containing 12.5 mM glucose, 5 ng/mL TNF-alpha, or other concentrations of glucose, TNF-alpha, or other sugars and/or cytokines.

In some cases, combinations of biomarkers are used in the subject methods. In some such cases, the levels of all measured biomarkers must change (as described above) in order for the determination to be made that the individual is responsive, maintaining responsiveness, or not responsive to a pre-diabetic treatment. In some embodiments, phosphorylation of Akt, levels of reactive oxygen species (ROS), levels of NO, and levels of expression of SIRT1, eNOS, CDH13, and IRS1 are used for evaluating progression of prediabetes in the subject, wherein detection of reduced phosphorylation of Akt, increased levels of ROS, decreased levels of NO, decreased levels of expression of SIRT1 and IRS1, and increased levels of expression of eNOS and CDH13 in the IPSC-ECs derived from the somatic cells of the second sample compared to the IPSC-ECs derived from the somatic cells of the first sample indicates that the subject is worsening or not responding to the therapy, and detection of increased phosphorylation of Akt, decreased levels of ROS, increased levels of NO, increased levels of expression of SIRT1 and IRS1, and decreased levels of expression of eNOS and CDH13 in the IPSC-ECs derived from the somatic cells of the second sample compared to the IPSC-ECs derived from the somatic cells of the first sample indicates that the subject is improving.

In some embodiments, only some biomarkers selected from phosphorylated Akt, ROS, and NO production, and expression of SIRT1, IRS1, eNOS and CDH13 are used in the methods described herein. For example, a single biomarker, two types of biomarkers, three types of biomarkers, four types of biomarkers, or five types of biomarkers can be used in any combination. In other embodiments, all the biomarkers are used. The quantitative values may be combined in linear or non-linear fashion to calculate one or more risk scores for insulin resistance risks for the individual.

The level of a biomarker in IPSC-ECs derived from somatic cells of a pre-treatment biological sample can be referred to as a “pre-treatment value” because the first biological sample is isolated from the individual prior to the administration of the pre-diabetic therapy (i.e., “pre-treatment”). The level of a biomarker in the IPSC-ECs derived from the somatic cells of the pre-treatment biological sample can also be referred to as a “baseline value” because this value is the value to which “post-treatment” values are compared. In some cases, the baseline value (i.e., “pre-treatment value”) is determined by determining the level of a biomarker from IPSC-ECs derived from the somatic cells of multiple (i.e., more than one, e.g., two or more, three or more, for or more, five or more, etc.) pre-treatment biological samples. In some cases, the multiple pre-treatment biological samples are isolated from an individual at different time points in order to assess natural fluctuations in biomarker levels prior to treatment. As such, in some cases, one or more (e.g., two or more, three or more, for or more, five or more, etc.) pre-treatment biological samples comprising somatic cells are isolated from the individual. In some embodiments, all of the pre-treatment biological samples will be the same type of biological sample (e.g., a blood sample, a biopsy sample, etc.). In some cases, two or more pre-treatment biological samples are pooled prior to determining the level of the biomarker in the IPSC-ECs derived from the somatic cells of the biological samples. In some cases, the level of the biomarker is determined separately for IPSC-ECs derived from the somatic cells of two or more pre-treatment biological samples and a “pre-treatment value” is calculated by averaging the separate measurements.

A post-treatment biological sample is isolated from an individual after the administration of a pre-diabetic therapy. Thus, the level of a biomarker in IPSC-ECs derived from the somatic cells of a post-treatment sample can be referred to as a “post-treatment value”. In some embodiments, the level of a biomarker is measured in IPSC-ECs derived from the somatic cells of additional post-treatment biological samples (e.g., a second, third, fourth, fifth, etc. post-treatment biological sample). Because additional post-treatment biological samples are isolated from the individual after the administration of a pre-diabetic treatment, the levels of a biomarker in the IPSC-ECs derived from the somatic cells of additional biological samples can also be referred to as “post-treatment values.”

The term “responsive” as used herein means that the pre-diabetic treatment is having the desired effect and the treatment is decreasing the risk of the individual of developing insulin resistance. When the individual does not improve in response to the pre-diabetic treatment, it may be desirable to seek a different therapy or treatment regime for the individual.

The determination that an individual is at risk of developing insulin resistance is a direct and active clinical application of the correlation between levels of a biomarker and the risk of developing insulin resistance. For example, “determining” requires the active step of reviewing the data, which is produced during the active assaying step(s), and resolving whether an individual is or is not at risk of developing insulin resistance or responding to a pre-diabetic treatment to decrease the risk of developing insulin resistance and type 2 diabetes. Additionally, in some cases, a decision is made to proceed with the current treatment (i.e., therapy), or instead to alter the treatment. In some cases, the subject methods include the step of continuing therapy or altering therapy.

The term “continue treatment” (i.e., continue therapy) is used herein to mean that the current course of treatment (e.g., continued administration of a pre-diabetic therapy) is to continue. If the current course of treatment is not effective in lowering the risk of developing insulin resistance and/or type 2 diabetes, the pre-diabetic treatment may be altered. “Altering therapy” is used herein to mean “discontinuing therapy” or “changing the therapy” (e.g., changing the type of pre-diabetic treatment, changing the particular dose and/or frequency of administration of medication, e.g., increasing the dose and/or frequency). In some cases, therapy can be altered until the individual is deemed to be responsive. In some embodiments, altering therapy means changing which type of pre-diabetic treatment is administered, discontinuing a particular treatment altogether, etc.

As a non-limiting illustrative example, a patient may be initially treated by putting the patient on a diet that reduces carbohydrate intake, and asking the patient to lose weight and increase exercise. Then to “continue treatment” would be to continue with these types of treatments. If the current course of treatment is not effective in lowering the risk of developing insulin resistance and/or type 2 diabetes, the pre-diabetic treatment may be altered, e.g., increasing amount of exercise, increasing weight loss with stricter diet or bariatric surgery, administering metformin to a subject at high risk of developing insulin resistance.

In other words, the level of one or more biomarkers may be monitored in order to determine when to continue therapy and/or when to alter therapy. As such, a post-treatment biological sample can be isolated after any of the administrations and IPSC-ECs derived from the somatic cells of the biological sample can be assayed to determine the level of a biomarker. Accordingly, the subject methods can be used to determine whether an individual being treated for pre-diabetic symptoms and/or symptoms of insulin resistance is responsive or is maintaining responsiveness to a pre-diabetic treatment.

The pre-diabetic therapy can be administered to an individual any time after a pre-treatment biological sample is isolated from the individual, but it is preferable for the pre-diabetic therapy to be administered simultaneous with or as soon as possible (e.g., about 7 days or less, about 3 days or less, e.g., 2 days or less, 36 hours or less, 1 day or less, 20 hours or less, 18 hours or less, 12 hours or less, 9 hours or less, 6 hours or less, 3 hours or less, 2.5 hours or less, 2 hours or less, 1.5 hours or less, 1 hour or less, 45 minutes or less, 30 minutes or less, 20 minutes or less, 15 minutes or less, 10 minutes or less, 5 minutes or less, 2 minutes or less, or 1 minute or less) after a pre-treatment biological sample is isolated (or, when multiple pre-treatment biological samples are isolated, after the final pre-treatment biological sample is isolated).

In some cases, more than one type of pre-diabetic therapy may be administered to the individual. For example, a subject may be put on a reduced carbohydrate diet. Subjects having a sedentary lifestyle may be advised to also increase exercise. Additionally, bariatric surgery may be performed on obese subjects to assist weight loss in addition to reducing carbohydrate intake and increasing exercise. Metformin may be further administered to subjects at high risk of developing type 2 diabetes. In addition, subjects may be administered medication to treat high blood and/or high cholesterol. The pre-diabetic treatment may further comprise administering to the subject a sulfonylurea, a thiazolidinedione, a dipeptidyl peptidase-4 inhibitor, a sodium-glucose transport protein 2 (SGLT2) inhibitor, a glucagon-like peptide-1 analog, an angiotensin-converting enzyme inhibitor (ACEI), or a combination thereof.

In some embodiments, the subject methods include providing an analysis indicating whether the individual is determined to be at risk of developing insulin resistance. The analysis may further provide an analysis of whether an individual is responsive or not responsive to a pre-diabetic treatment, or whether the individual is determined to be maintaining responsiveness or not maintaining responsiveness to a pre-diabetic treatment. As described above, an analysis can be an oral or written report (e.g., written or electronic document). The analysis can be provided to the subject, to the subject's physician, to a testing facility, etc. The analysis can also be accessible as a website address via the internet. In some such cases, the analysis can be accessible by multiple different entities (e.g., the subject, the subject's physician, a testing facility, etc.).

### Detecting and Measuring Biomarkers

It is understood that the biomarkers in a sample can be measured by any suitable method known in the art. Measurement of the expression level of a biomarker can be direct or indirect. For example, the abundance levels of RNAs or proteins can be directly quantitated. Alternatively, the amount of a biomarker can be determined indirectly by measuring abundance levels of cDNAs, amplified RNAs or DNAs, or by measuring quantities or activities of RNAs, proteins, or other molecules (e.g., metabolites or metabolic byproducts) that are indicative of the expression level of the biomarker. The methods for measuring biomarkers in a sample have many applications. For example, one or more biomarkers can be measured to aid in evaluating the risk of a subject of developing insulin resistance and determining the appropriate treatment for a subject, as well as monitoring responses of a subject to treatment.

In some embodiments, the amount or level in the sample of one or more proteins/polypeptides encoded by a gene of interest is determined. Any convenient protocol for evaluating protein levels may be employed where the level of one or more proteins in the assayed sample is determined. For antibody-based methods of protein level determination, any convenient antibody can be used that specifically binds to the intended biomarker (e.g., phosphorylated Akt, sirtuin 1 (SIRT1), endothelial nitric oxide synthase (eNOS), cadherin 13 (CDH13), and insulin receptor substrate 1 (IRS1)). The terms “specifically binds” or “specific binding” as used herein refer to preferential binding to a molecule relative to other molecules or moieties in a solution or reaction mixture (e.g., an antibody specifically binds to a particular polypeptide or epitope relative to other available polypeptides or epitopes). In some embodiments, the affinity of one molecule for another molecule to which it specifically binds is characterized by a K_{d} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). By “Affinity” it is meant the strength of binding, increased binding affinity being correlated with a lower K_{d}.

While a variety of different manners of assaying for protein levels are known in the art, one representative and convenient type of protocol for assaying protein levels is the enzyme-linked immunosorbent assay (ELISA). In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Such conditions include diluting the sample with diluents such as BSA or bovine gamma globulin (BGG) in phosphate buffered saline (PBS)/Tween or PBS/Triton-X 100, which also tend to assist in the reduction of nonspecific background, and allowing the sample to incubate for about 2-4 hours at temperatures on the order of about 25°−27° C. (although other temperatures may be used). Following incubation, the antisera-contacted surface is washed so as to remove non-immunocomplexed material. An exemplary washing procedure includes washing with a solution such as PBS/Tween, PBS/Triton-X 100, or borate buffer. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. For example, a urease or peroxidase-conjugated anti-human IgG may be employed, for a period of time and under conditions which favor the development of immunocomplex formation (e.g., incubation for 2 hours at room temperature in a PBS-containing solution such as PBS/Tween). After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H₂O2, in the case of a peroxidase label. Quantitation is then achieved by measuring the degree of color generation, e.g., using a visible spectrum spectrophotometer. The preceding format may be altered by first binding the sample to the assay plate. Then, primary antibody is incubated with the assay plate, followed by detecting of bound primary antibody using a labeled second antibody with specificity for the primary antibody.

The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatographic column or filter with a wash solution or solvent.

Alternatively, non-ELISA based-methods for measuring the levels of one or more proteins in a sample may be employed and any convenient method may be used. Representative examples known to one of ordinary skill in the art include but are not limited to other immunoassay techniques such as radioimmunoassays (RIA), sandwich immunoassays, fluorescent immunoassays, enzyme multiplied immunoassay technique (EMIT), capillary electrophoresis immunoassays (CEIA), and immunoprecipitation assays; mass spectrometry, or tandem mass spectrometry, proteomic arrays, xMAP microsphere technology, western blotting, immunohistochemistry, flow cytometry, cytometry by time-of-flight (CyTOF), multiplexed ion beam imaging (MIBI), and detection in body fluid by electrochemical sensor. In, for example, flow cytometry methods, the quantitative level of gene products of the one or more genes of interest are detected on cells in a cell suspension by lasers. As with ELISAs and immunohistochemistry, antibodies (e.g., monoclonal antibodies) that specifically bind the polypeptides encoded by the genes of interest are used in such methods.

As another example, electrochemical sensors may be employed. In such methods, a capture aptamer or an antibody that is specific for a target protein (the “analyte”) is immobilized on an electrode. A second aptamer or antibody, also specific for the target protein, is labeled with, for example, pyrroquinoline quinone glucose dehydrogenase ((PQQ)GDH). The sample of body fluid is introduced to the sensor either by submerging the electrodes in body fluid or by adding the sample fluid to a sample chamber, and the analyte allowed to interact with the labeled aptamer/antibody and the immobilized capture aptamer/antibody. Glucose is then provided to the sample, and the electric current generated by (PQQ)GDH is observed, where the amount of electric current passing through the electrochemical cell is directly related to the amount of analyte captured at the electrode.

Flow cytometry can be used to distinguish subpopulations of cells expressing different cellular markers and to determine their frequency in a population of cells. Typically, whole cells are incubated with antibodies that specifically bind to the cellular markers. The antibodies can be labeled, for example, with a fluorophore, isotope, or quantum dot to facilitate detection of the cellular markers. The cells are then suspended in a stream of fluid and passed through an electronic detection apparatus. In addition, fluorescence-activated cell sorting (FACS) can be used to sort a heterogeneous mixture of cells into separate containers. (See, e.g., Shapiro Practical Flow Cytometry, Wiley-Liss, 4^{th} edition, 2003; Loken Immunofluorescence Techniques in Flow Cytometry and Sorting, Wiley, 2^{nd} edition, 1990; Flow Cytometry: Principles and Applications, (ed. Macey), Humana Press 1^{st} edition, 2007; herein incorporated by reference in their entireties.)

Cytometry by time-of-flight (CyTOF), also known as mass cytometry, is a method that can be used for detection of cellular markers in whole cells. CyTOF uses transition element isotopes as labels for antibodies, which are detected by a time-of-flight mass spectrometer. Unlike conventional flow cytometry, CyTOF is destructive to cells, but has the advantage that it can be used to analyze more cell markers simultaneously. See, e.g., Bendall et al. (2012) Trends in Immunology 33:323-332; Newell et al. (2012) Immunity 36(1):142-52; Ornatsky et al. (2010) J. Immunol. Methods 361 (1-2):1-20; Bandura et al. (2009) Analytical Chemistry 81:6813-6822; Chen et al. (2012) Cell Mol. Immunol. 9(4):322-323; and Cheung et al. (2011) Nat. Rev. Rheumatol. 7(9):502-3; herein incorporated by reference in their entireties.

In addition, multiplexed ion beam imaging (MIBI) can be used to distinguish subpopulations of cells carrying different cellular markers. MIBI uses secondary ion mass spectrometry to image antibodies that are tagged with isotopically pure elemental metal reporters. Not only can MIBI measure protein levels on individual cells, but also, the technique is capable of providing information about cell morphology and localization. Like CYTOF, MIBI is capable of analyzing a large number of cell markers (e.g., up to 100) simultaneously over a five-log dynamic range. See, e.g., Angelo et al. (2014) Nat. Med. 20(4):436-442; Bodenmiller et al. (2016) Cell Syst. 2(4):225-238; and Levenson et al. (2015) Lab Invest. 95(4):397-405; herein incorporated by reference in their entireties.

For measuring protein activity levels, the amount or level of protein activity in the sample of one or more proteins/polypeptides encoded by the gene of interest is determined.

In other embodiments, the amount or level in the sample of one or more RNAs encoded by a gene of interest is determined. Any convenient method for measuring mRNA levels in a sample may be used, e.g. hybridization-based methods, e.g. northern blotting and in situ hybridization (Parker & Barnes, Methods in Molecular Biology 106:247-283 (1999)), RNase protection assays (Hod, Biotechniques 13:852-854 (1992)), and PCR-based methods (e.g. reverse transcription PCR (RT-PCR) (Weis et al., Trends in Genetics 8:263-264 (1992)). Alternatively, any convenient method for measuring protein levels in a sample may be used, e.g. antibody-based methods, e.g. immunoassays, e.g., enzyme-linked immunosorbent assays (ELISAs), immunohistochemistry, and flow cytometry (FACS).

For measuring mRNA levels, the starting material may be total RNA, i.e. unfractionated RNA, or poly A+ RNA isolated from a suspension of cells (e.g. IPSC-ECs). General methods for mRNA extraction are well known in the art and are disclosed in standard textbooks of molecular biology, including Ausubel et al., Current Protocols of Molecular Biology, John Wiley and Sons (1997). RNA isolation can also be performed using a purification kit, buffer set and protease from commercial manufacturers, according to the manufacturer's instructions. For example, RNA from cell suspensions can be isolated using Qiagen RNeasy mini-columns, and RNA from cell suspensions or homogenized tissue samples can be isolated using the TRIzol reagent-based kits (Invitrogen), MasterPure™ Complete DNA and RNA Purification Kit (EPICENTRE™, Madison, Wis.), Paraffin Block RNA Isolation Kit (Ambion, Inc.) or RNA Stat-60 kit (Tel-Test).

The mRNA levels may be measured by any convenient method. Examples of methods for measuring mRNA levels may be found in, e.g., the field of differential gene expression analysis. One representative and convenient type of protocol for measuring mRNA levels is array-based gene expression profiling. Such protocols are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively.

Specific hybridization technology which may be employed in the subject methods includes that described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed. The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

The resultant pattern of hybridized nucleic acid provides information regarding expression for each of the genes that have been probed, where the expression information is in terms of whether or not the gene is expressed and, typically, at what level, where the expression data, i.e., expression profile (e.g., in the form of a transcriptosome), may be both qualitative and quantitative.

Additionally or alternatively, non-array based methods for quantitating the level of one or more nucleic acids in a sample may be employed. These include those based on amplification protocols, e.g., Polymerase Chain Reaction (PCR)-based assays, including quantitative PCR, reverse-transcription PCR (RT-PCR), real-time PCR, and the like, e.g. TaqMan, RT-PCR, MassARRAY System, BeadArray technology, and Luminex technology; and those that rely upon hybridization of probes to filters, e.g. Northern blotting and in situ hybridization. Serial Analysis Gene Expression (SAGE) can also be used to determine RNA abundances in a cell sample. See, e.g., Velculescu et al., 1995, Science 270:484-7; Carulli, et al., 1998, Journal of Cellular Biochemistry Supplements 30/31:286-96; herein incorporated by reference in their entireties. SAGE analysis does not require a special device for detection, can be used for simultaneously detecting the expression of large numbers of transcription products.

In some embodiments, the amount of ROS produced in the IPSC-ECs is detected. One or more types of ROS may be detected including, without limitation, peroxides, superoxide, hydroxyl radical, singlet oxygen, and alpha-oxygen, which are produced as a byproduct of the metabolism of oxygen in cells. Such ROS may be detected by any method known in the art. For example, a cell-permeable fluorogenic probe (e.g., DCFH-DA, CeIIROX Green) can be used to assay intracellular ROS. The dyes are nonfluorescent in a reduced state but fluoresce upon oxidation by ROS. Alternatively, ROS can be detected with the ROS-Glo H₂O₂ assay (Promega Corporation, Madison, Wis.), which is a luminescent assay that detects H₂O₂ using luciferase to generate a light signal proportional to the level of H₂O₂ present.

In some embodiments, the amount of NO produced by the IPSC-ECs is detected. For example, NO levels may be detected using the Griess assay which measures nitrate and nitrite in correlation with NO release. NO may also be detected using a fluorescent dye such as diaminofluorescein (DAF) or its cell-permeable derivatives (e.g., DAF-2DA or DAF-FM DA), which react with NO to form an intensely fluorescent triazole derivative. Fluorescence intensity is proportional to the amount of NO produced in cells. See, e.g., Itoh et al. (2000) Anal. Biochem. 287(2):203-9, Kojima et al. (1998) Anal Chem. 70(13):2446-53; herein incorporated by reference.

The resultant data provides information regarding expression, amount, and/or activity for each of the biomarkers that have been measured, wherein the information is in terms of whether or not the biomarker is present (e.g. expressed) and at what level, and wherein the data may be both qualitative and quantitative.

### Data Analysis

In some embodiments, one or more pattern recognition methods can be used in analyzing the data for biomarker levels. The quantitative values may be combined in linear or non-linear fashion to calculate one or more risk scores for insulin resistance risks for an individual. In some embodiments, measurements for a biomarker or combinations of biomarkers are formulated into linear or non-linear models or algorithms (e.g., a ‘biomarker signature’) and converted into a likelihood score. This likelihood score indicates the probability that a biological sample is from a patient who may exhibit no evidence of disease, who may exhibit a risk of developing insulin resistance, or who may exhibit insulin resistance. The likelihood score can be used to distinguish these disease states. The models and/or algorithms can be provided in machine readable format, and may be used to correlate biomarker levels or a biomarker profile with a disease state, and/or to designate a treatment modality for a patient or class of patients.

Analyzing the levels of a plurality of biomarkers may comprise the use of an algorithm or classifier. In some embodiments, a machine learning algorithm is used to classify a patient as being at risk of developing insulin resistance. For example, a machine learning algorithm may be used for computing a risk score for insulin resistance. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

Preferably, the machine learning algorithms may include, but are not limited to, Average One-Dependence Estimators (AODE), Fisher's linear discriminant, Logistic regression, Perceptron, Multilayer Perceptron, Artificial Neural Networks, Support vector machines, Quadratic classifiers, Boosting, Decision trees, C4.5, Bayesian networks, Hidden Markov models, High-Dimensional Discriminant Analysis, and Gaussian Mixture Models. The machine learning algorithm may comprise support vector machines, Naïve Bayes classifier, k-nearest neighbor, high-dimensional discriminant analysis, or Gaussian mixture models. In some instances, the machine learning algorithm comprises Random Forests.

### Kits

Also provided are kits for use in the methods. The subject kits include agents (e.g., a PCR primer pair or a probe specific for detecting the level of expression of a biomarker, an antibody that specifically binds to a biomarker and/or other immunoassay reagents, bioluminescent assay reagents for measuring ROS, and the like) for determining the level of at least one biomarker. In some embodiments, a kit comprises agents for determining the level of a single biomarker, two or more different biomarkers, or all the biomarkers selected from the group consisting of phosphorylated Akt, ROS, NO, SIRT1, eNOS, CDH13, and IRS1. In some embodiments, the kit further comprises agents for producing IPSC-ECs from a patient's somatic cells (e.g., Yamanaka factors and differentiation agents such as VEGF, bFGF, and BMP4. In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, flash drive, and the like, on which the information has been recorded. Yet another form of these instructions that may be present is a website address which may be used via the internet to access the information at a removed site.

It will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

An iPSC Model of Insulin Resistance Predicts Type 2 Diabetes

### Introduction

Insulin resistance is central to the development of type 2 diabetes (T2DM)¹ and presents a major risk factor for cardiovascular disease^{2,3}. South Asians have up to four-fold higher incidence of insulin resistance age-matched Europeans⁴⁻⁶ that cannot be adequately explained by environmental factors such as diet and sedentary lifestyle. Insulin resistance is difficult to diagnose prior to the onset of T2DM and is often underdiagnosed⁷, with the prevalence of insulin resistance estimated to be three times greater than T2DM⁸. The causes of insulin resistance and how it impacts the vascular system are incompletely understood. Therefore, there is a need to identify early diagnostic strategies for insulin resistance and potential therapeutic targets.

We examined the molecular mechanisms of how insulin resistance causes vascular dysfunction using induced pluripotent stem cells (iPSCs) derived endothelial cells (iPSC-ECs). Endothelial cells line blood vessels and capillaries, and serve as the first interface between the cardiovascular system and blood circulating factors such as glucose, insulin, and inflammatory cytokines. Notably, iPSC-EC models have the ability to generate abundant patient-specific tissue samples that may more aptly reflect underlying individual susceptibility to atherosclerosis than animal models^{9,10}. Moreover, iPSC-ECs provide an excellent platform to investigate the molecular mechanisms of insulin resistance and cardiovascular diseases as they retain the genetic information of the individuals while erasing their epigenetic environment during reprogramming¹¹. Using this platform, we observed several novel phenotypic features of insulin resistance in the donor cohort and devised an iPSC risk scoring calculator for insulin resistance.

### Results

### Insulin Resistance Patients Exhibit Endothelial Dysfunction

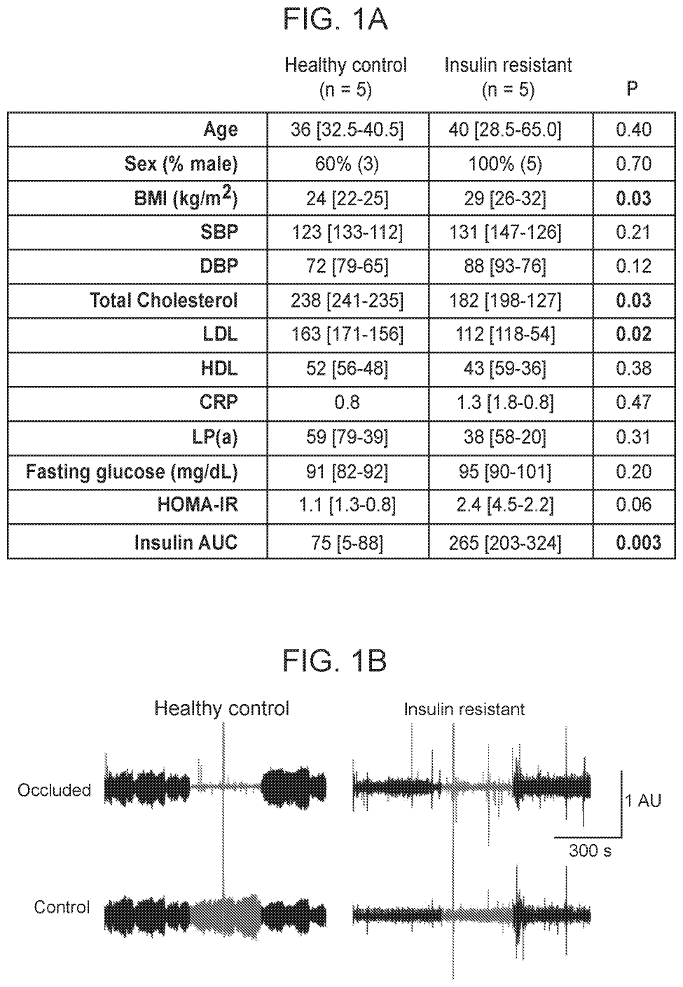

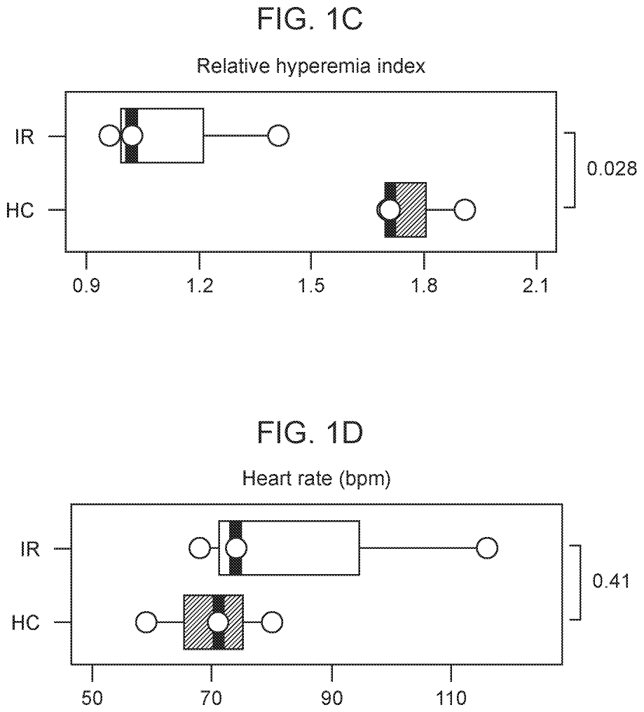

We identified and recruited five South Asian insulin-resistant and five healthy control patients using an oral glucose tolerance test (FIG. 1A and Table 1). The patients were challenged with an oral glucose load and serum measurements of insulin and glucose were obtained every thirty minutes until glucose and insulin returned to baseline¹². A normal fasting glucose and a markedly elevated insulin area under the curve (AUC) define insulin resistance. Baseline data include age, sex, fasting glucose and insulin AUC required for glucose disposal are summarized. Patient demographics reveal no difference in age or sex, but insulin-resistant patients have a higher BMI.

Endothelial dysfunction is known to precede the development of obstructive coronary disease¹³. The gold standard for endothelial functional assessment is the coronary angiogram with provocative acetylcholine testing, but the procedure can be invasive and carry potential complications compared to non-invasive assessments such as flow mediated dilatation and the reactive hyperemia peripheral arterial tonometry. In particular, reactive hyperemia peripheral artery tonometry is automated and has low interobserver variability. We therefore measured the endothelial function of insulin-resistant patients using peripheral arterial tonometry. The participants were monitored with an endoPAT instrument at baseline for five minutes before occlusion of the non-dominant arm for five minutes (FIG. 1B). Reactive hyperemia was induced after releasing the cuff which was then calculated as the reactive hyperemia index (RHI) by endoPAT software. Remarkably, insulin-resistant participants showed abnormal hyperemia index at baseline when compared to normal healthy controls (1.8 vs. 1.1, t-test p=0.03, FIG. 1C) with comparable heart rates (FIG. 1D), suggesting insulin-resistant patients have abnormal vascular function.

Modeling Endothelial Dysfunction in iPSC-ECs with a Prediabetic Cocktail

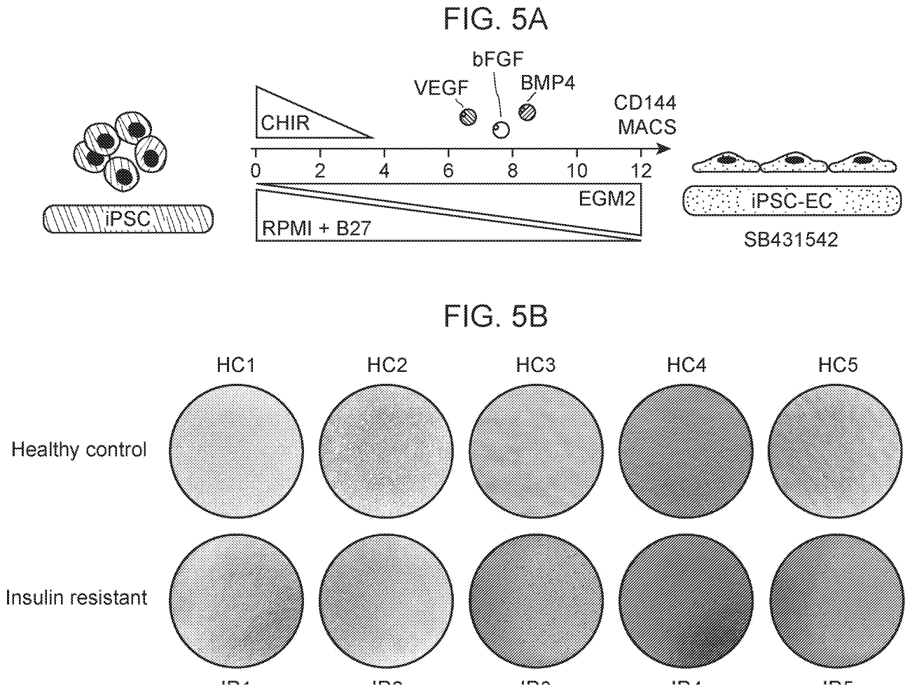

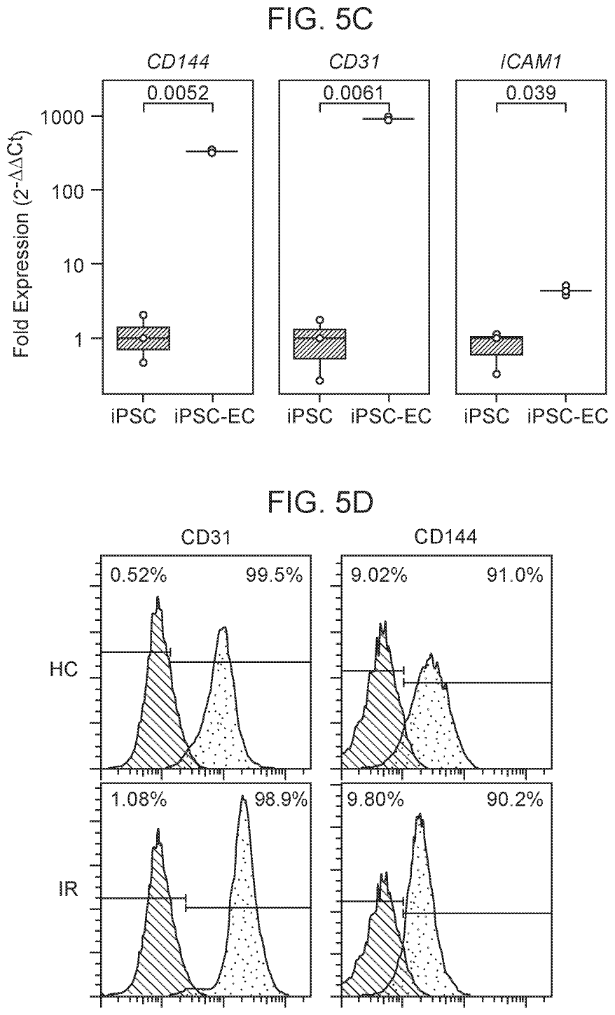

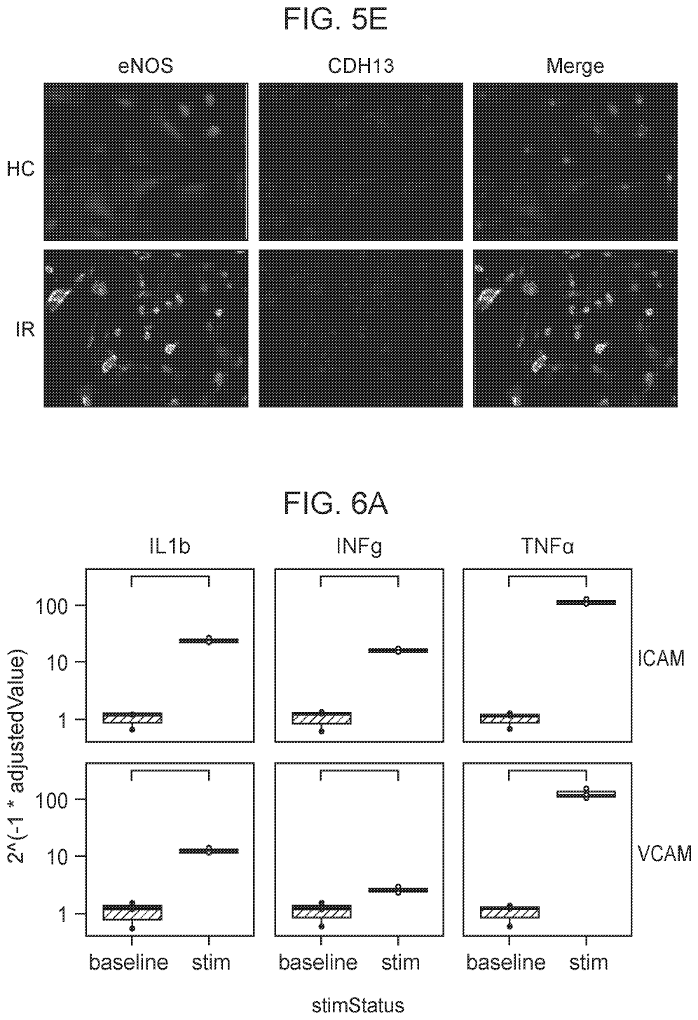

To investigate the mechanism behind endothelium dysfunction of the insulin-resistant individuals, we generated iPSC-ECs from peripheral blood mononuclear cells (PBMCs) of healthy control and insulin resistant patients. The PBMCs were reprogrammed to iPSCs based on our chemically-defined protocols^{14,15}. We then used an established endothelial differentiation protocol to differentiate these patient-specific iPSCs to endothelial cells (iPSC-ECs) (FIG. 5A)^{11,16}. The produced iPSC-ECs exhibited typical cobblestone-like morphologies (FIG. 5B) and showed high expression of endothelial markers CD31, CD144, and ICAM when measured by qPCR (FIG. 5C), fluorescence-activated cell sorting (FIG. 5D) and immunohistochemistry (FIG. 5E), suggesting they represent bona fide endothelial cells.

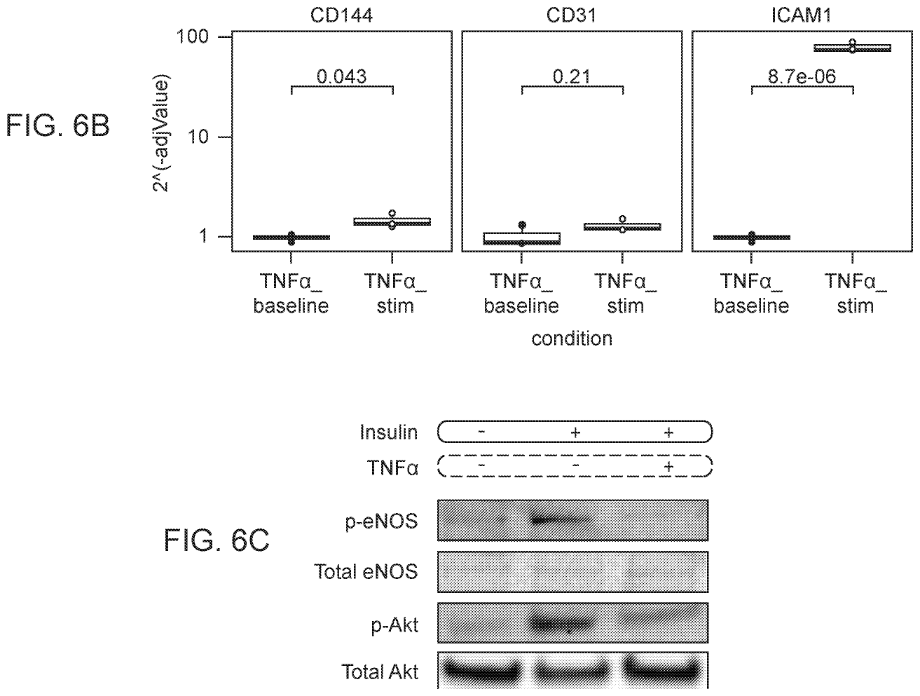

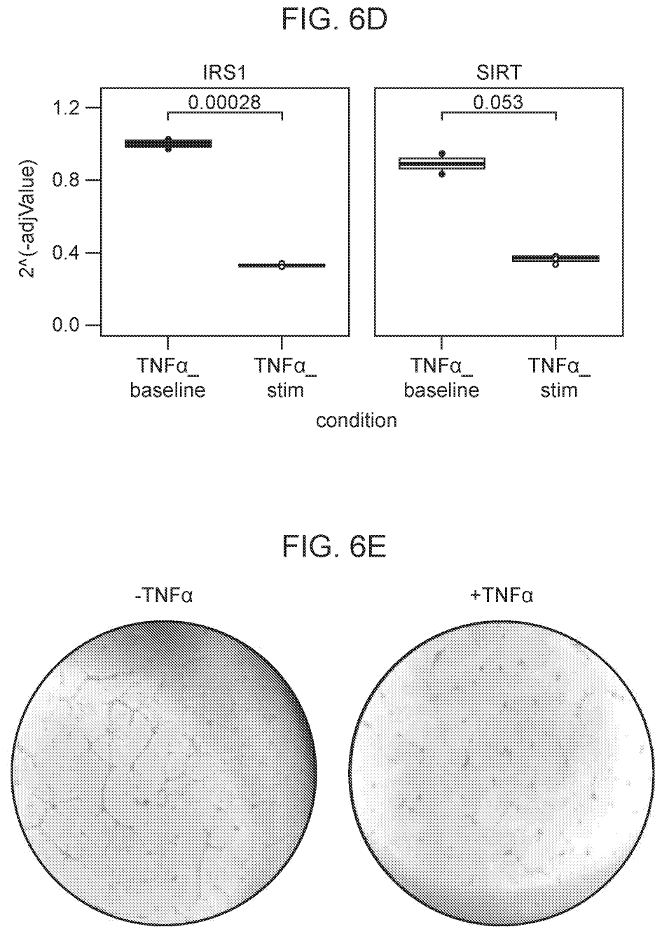

We next assessed whether the iPSC-ECs might serve as a model of insulin resistance by mimicking the paracrine effect of insulin resistance in vitro. Central obesity is associated with an increase in inflammatory cytokines^{17,18} and this inflammatory environment is implicated as the driving force in the development of insulin resistance^{19,20}. Low concentrations of cytokines have been shown to disrupt insulin signaling and mimic low levels of inflammation in cultured endothelial cells L²¹. We observed that exposure of low doses TNFα, IL1-β and INFγ strongly induced the expression of cell surface adhesion molecules including ICAM1 and VCAM1 (FIG. 6A). The expression changes are not explained by endothelial mesenchymal transition induced by the cytokines, because the levels of EC marker CD31 and CD144 levels and not significantly changed following TNFα exposure (FIG. 6B), suggesting the observed gene expression is likely due to proinflammatory effects mimicking insulin resistance.

Endothelial cell insulin receptors are known to be activated following insulin. The signaling cascade phosphorylates Akt and ultimately phosphorylates eNOS which causes NO release²². Following insulin stimulation, we observed that both eNOS and Akt are phosphorylated in the iPSC-ECs (FIGS. 6C and 6D). We found that TNFα disrupted insulin signaling in iPSC-ECs by attenuating phosphorylation of the insulin signaling cascade, as well as the expression of IRS1, a component of the insulin signaling pathway (FIG. 6E). Moreover, TNFα also diminished the expression of the insulin sensitizer, sirtuin 1 (SIRT1), which is implicated in NO production thus controlling reactive oxygen species (ROS) and maintaining vascular homeostasis 2. At 10 ng/mL, TNFα impairs angiogenesis in healthy iPSC-ECs (FIG. 6F). Taken together, this attenuation of Akt and eNOS phosphorylation in iPSC-ECs following TNFα stimulation suggests impaired insulin signaling, which supports the rationale of modeling the inflammatory milieu of insulin resistance in vitro.

Insulin-Resistant iPSC-ECs Show Impaired Angiogenic Potential and NO Production

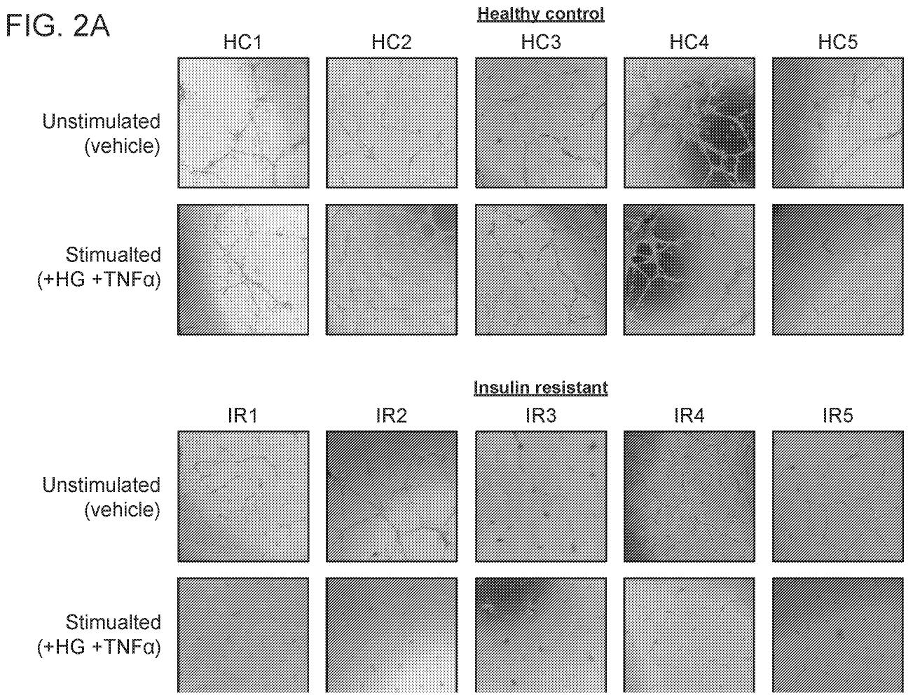

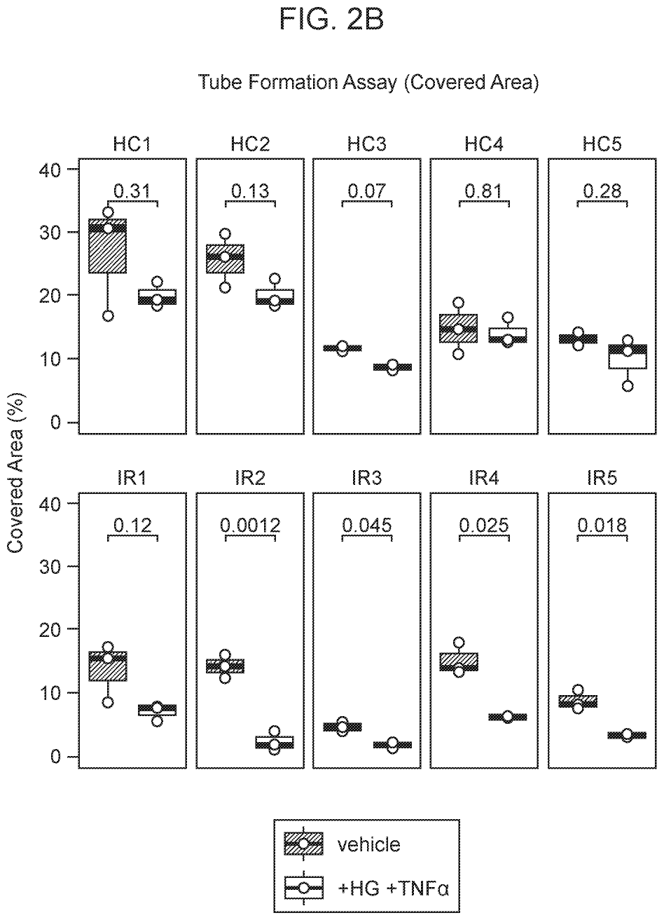

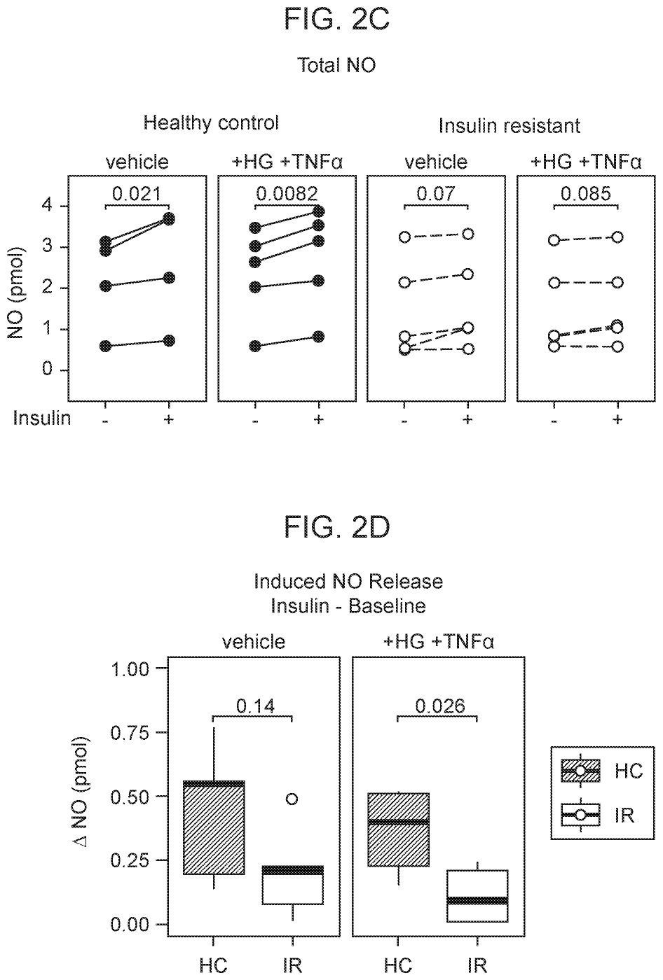

We next assessed the cellular phenotype of insulin-resistant iPSC-EC. We investigated the effects of perturbed gene expression in tube formation assays, which mimics angiogenesis in vitro. Tube formation assays revealed a significant decrease in each of covered area, branch points and tube length in insulin-resistant lines when treated with a prediabetic cocktail consisting of TNFα and high glucose (FIG. 2 and FIGS. 7A and 7B) but not in healthy control lines. In contrast, wound healing assays did not show a significant difference between healthy control and insulin-resistant lines (FIG. 7C).

To further evaluate the function of insulin resistance iPSC-ECs, we tested their NO release function using the Griess assay, which measures nitrate and nitrite that correlate with NO release²⁴⁻²⁶. Upon insulin stimulation, we observed an increase in NO production in healthy control iPSC-ECs. The release of NO by the cells was also confirmed using an alternative method with DAF-FM fluorescent dye (FIG. 7D). We saw that insulin-stimulated NO release was further pronounced under the prediabetic cocktail treatment (P=0.0082), but this response was attenuated in insulin-resistant iPSC-ECs (FIG. 2C). When directly comparing the inducible NO release (NO with insulin—NO at baseline) between healthy control and insulin-resistant iPSC-ECs, NO release was significantly lower in the prediabetic cocktail treated insulin resistant cells (P=0.026), indicating impaired NO production in these cells.

Biochemical and Gene Expression Analysis in Insulin-Resistant iPSC-ECs

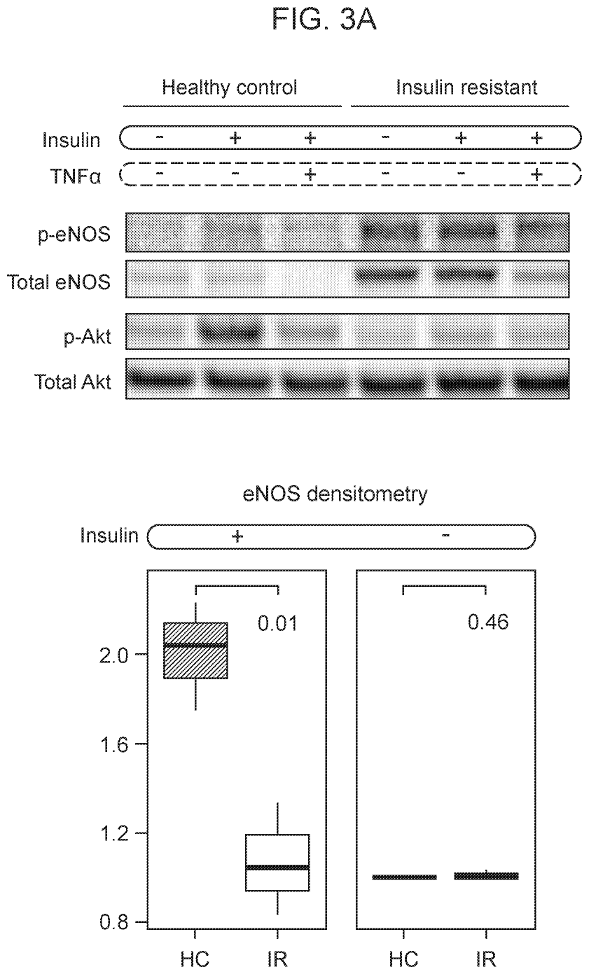

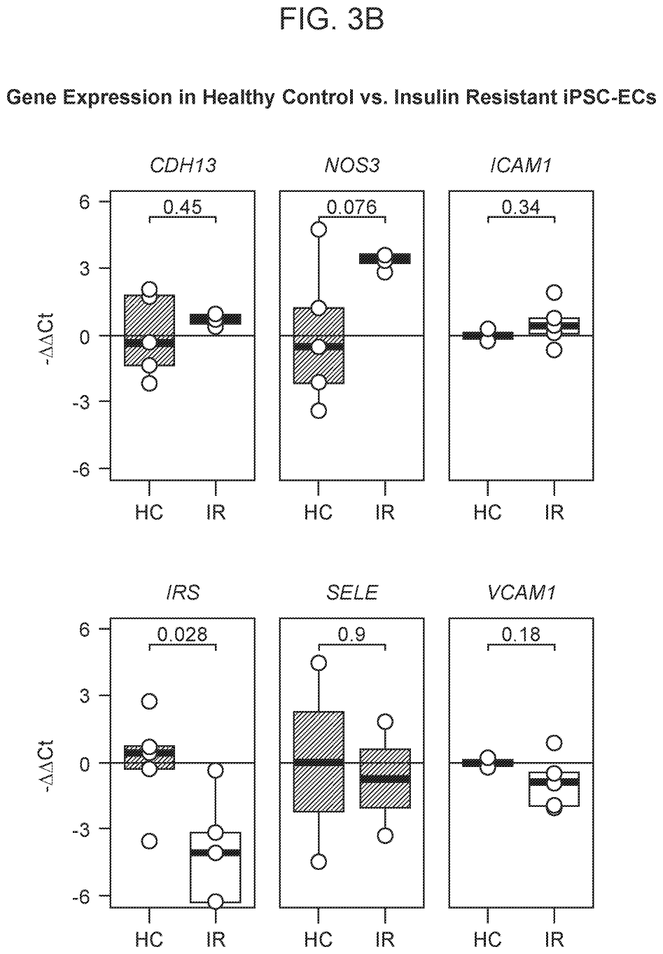

To understand these abnormalities, we investigated the protein expression and phosphorylation profile in insulin-resistant iPSC-ECs. As expected, healthy control iPSC-ECs expressed eNOS and Akt and both are phosphorylated in response to insulin. (FIG. 3A) The addition of TNFα disrupted insulin signaling emulating the effects of obesity and insulin resistance in an otherwise healthy individual. Insulin-resistant iPSC-ECs exhibited an increased expression of eNOS, which was phosphorylated at baseline without insulin stimulation. In contrast, when exposed to 100 nM insulin, insulin-resistant iPSC-ECs failed to exhibit a significant increase in Akt or eNOS phosphorylation when compared to controls. Moreover, TNFα attenuated phosphorylation of Akt and eNOS in healthy control and insulin-resistant lines. These observations were corroborated in an orthogonal analysis using ELISA for phosphorylated Akt. When normalized to total protein, Akt phosphorylation was reduced in insulin-resistant cells lines). When assessed by quantitative real time PCR, gene expression was perturbed in insulin-resistant lines. At baseline, SIRT1 was downregulated in insulin resistance lines, eNOS was elevated in all insulin-resistant lines and IRS1, a component of the insulin signaling cascade is downregulated.

Insulin resistance is associated with a variety of metabolic abnormalities which could be attributed to perturbed gene expression at baseline. To better understand these metabolic abnormalities, we screened known relevant insulin resistant genes in insulin-resistant iPSC-ECs (FIG. 3B).

Surprisingly, the expression of several genes implicated in insulin resistance and T2DM including insulin receptor (IR), tumour necrosis factor alpha receptor (TNFα) and forkhead binding protein 1 (FOXO1), E-selectin (SELE), cellular adhesion molecules (ICAM and VCAM) was not significantly difference when compared to healthy control. Instead, the quantitative real time PCR data showed that IR iPSC-ECs have altered gene expression for endothelial nitric oxide synthetase (NOS3), insulin receptor subunit 1 (IRS1) and adiponectin receptor 3 (CHD13). At baseline, NOS3 was elevated in all insulin resistant lines but not significantly and IRS1, a component of the insulin signaling cascade is downregulated (FIG. 3B). (CDH13) the receptor for adiponectin and implicated in insulin sensitivity is also upregulated in insulin resistant lines.

Unexpectedly, we found increased NOS3 expression in Insulin-resistant iPSC-EC at both the gene and protein level, despite impaired insulin signaling and consequently less NO production in the setting of increased ROS production. This phenotype might be due to an inherited factor that perturb gene expression. Our screen of genes implicated in insulin resistance and T2DM revealed that CHD13 might be an important player in altering NOS3 expression. Adiponectin is implicated in insulin resistance 2. In healthy individuals, adiponectin binds its cognate receptor on vascular cells, adiponectin receptor 3 (CDH13) and improves insulin signaling. In diabetes and obesity, adiponectin levels are low. Interestingly, overexpression of CDH13 in Koreans is associated with insulin resistance and cardiovascular disease. Moreover, overexpression of CDH13 in bovine coronary artery endothelial cells is known to increase eNOS expression and paradoxically cause an uncoupling of NO production, which causes endothelial dysfunction²⁹.

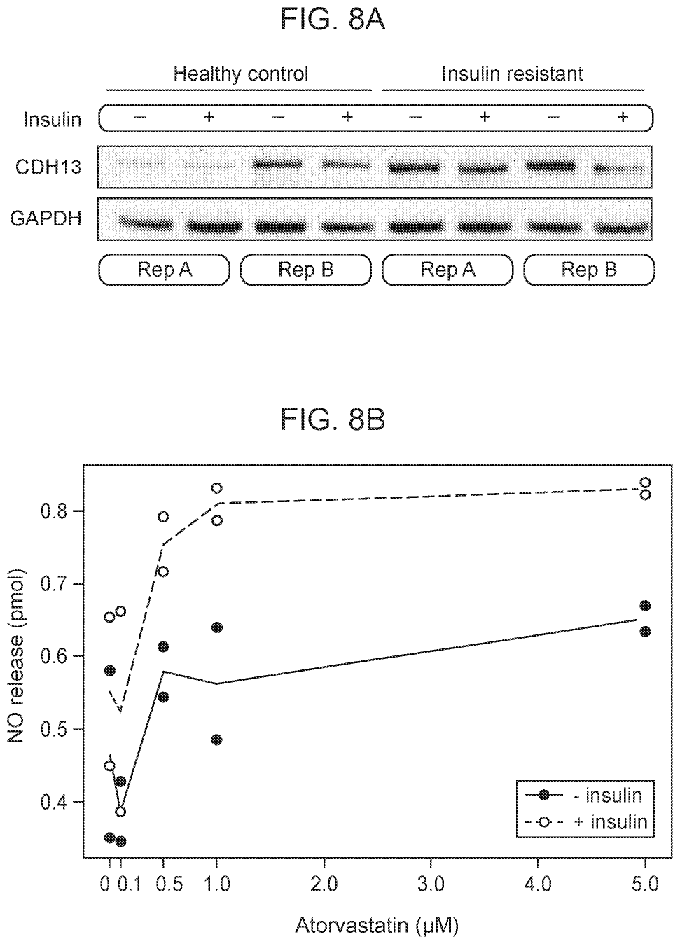

iPSC-ECs from insulin-resistant patients exhibit an increase in CDH13 expression (FIG. 8A). At the protein level, CDH13 expression is increased in insulin-resistant over healthy control and this effect was confirmed with densitometry. We did not detect any difference in other components of adiponectin signaling with insulin resistance. We next tested if CDH13 overexpression affected NOS3 expression in iPSC-ECs using siRNA directed against CDH13. When iPSC-ECs from healthy control or insulin-resistant patients were treated with siRNA, we found CDH13 expression was decreased by more than 85%. We also observed a concomitant decrease in NOS3 expression and surprisingly a significant increase in IRS1 expression. In the absence of a known small molecule inhibitor of CDH13, we wanted to investigate whether we could rescue eNOS uncoupling in insulin-resistant lines. Statins are known to increase eNOS expression³⁰ and atorvastatin is used for primary prevention of cardiovascular events in diabetics. We discovered that atorvastatin increased NO production in insulin resistant iPSC-ECs (FIG. 8B).

Risk Assessment of Insulin Resistance Using Individual-Specific iPSC-EC Measurements

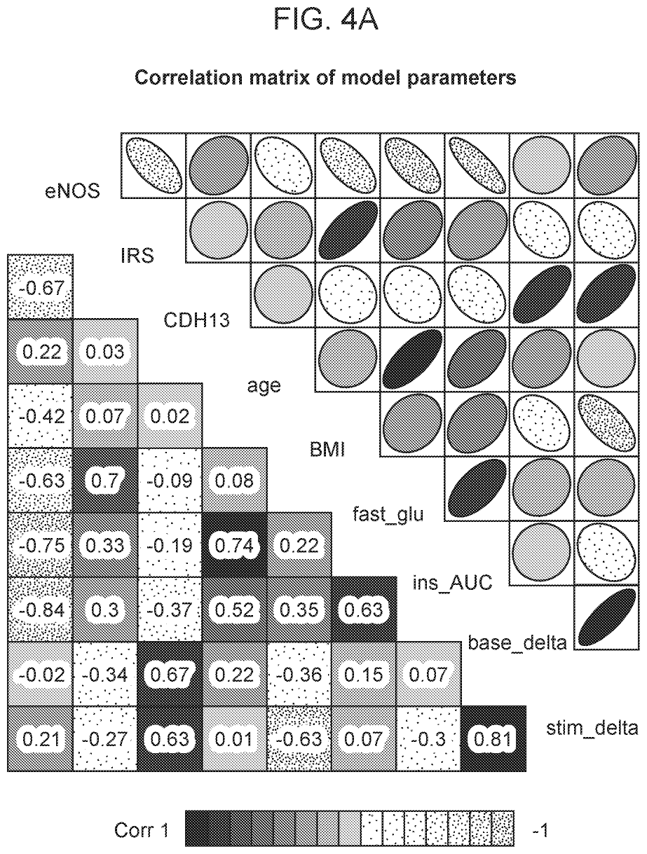

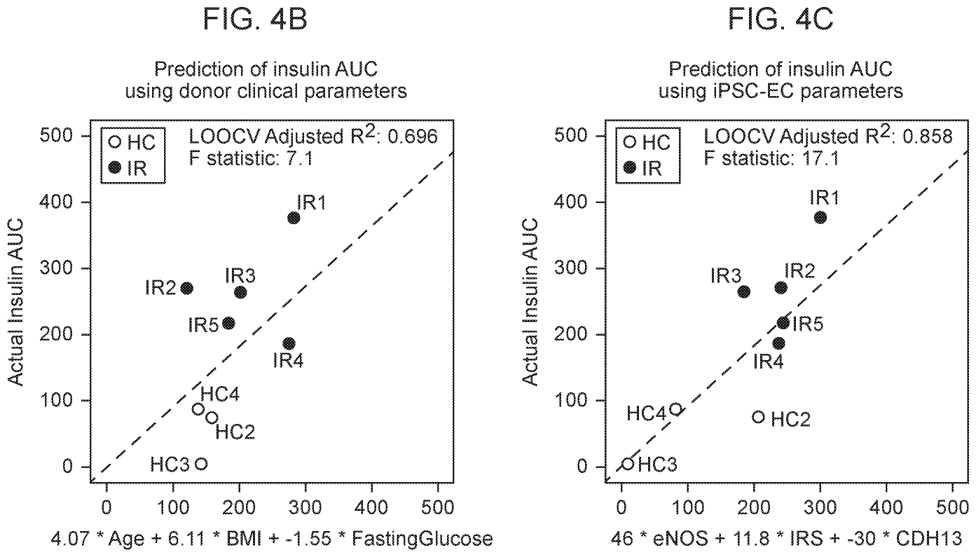

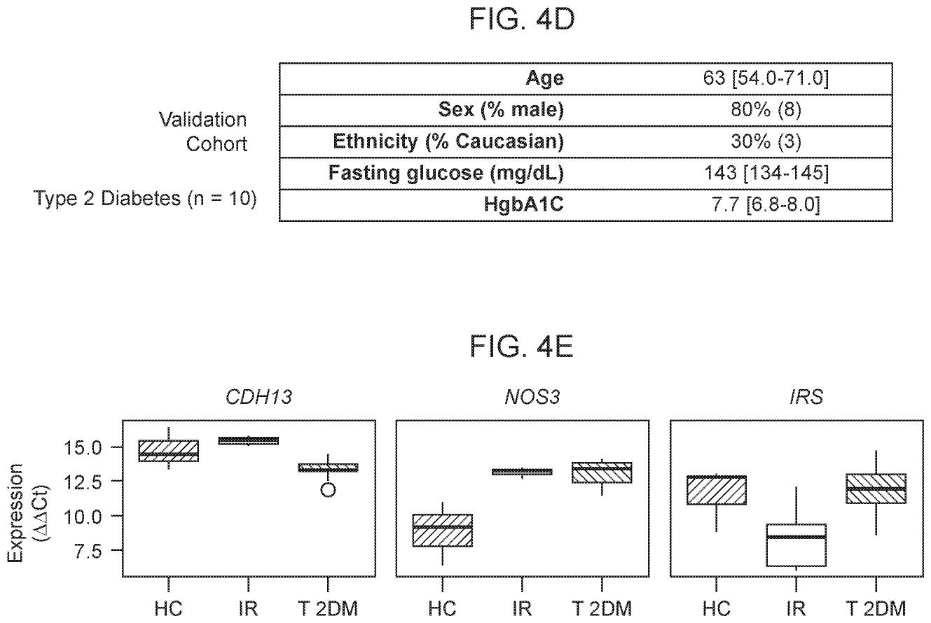

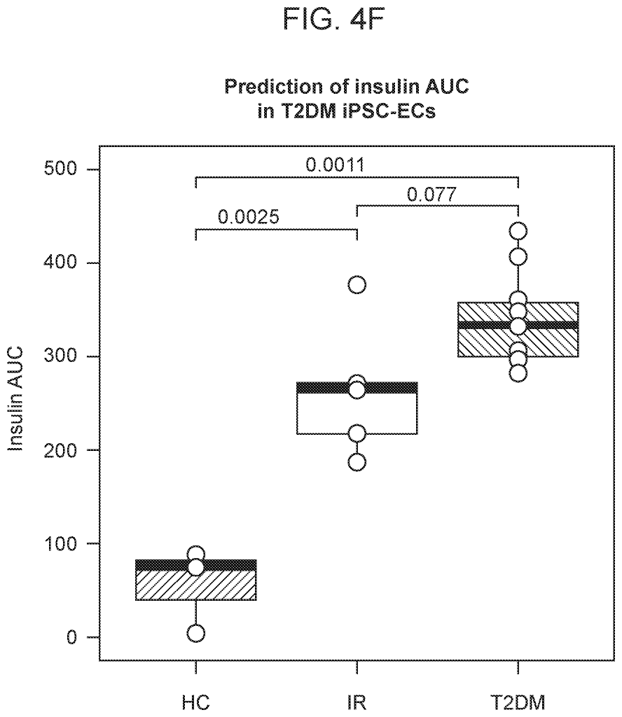

Effective means to identify individuals who are susceptible to insulin resistance could improve primary prevention through lifestyle change at a much earlier stage and avoid the need for drug therapy. At present however early diagnosis is not possible prior to the onset of observable phenotypes and results from genetic testing while promising have been inconclusive. We therefore sought to assess the feasibility of integrating multiple independent iPSC-EC observations (FIG. 4A) to discriminate between insulin-resistant and insulin-sensitive iPSC lines reported above to predict the insulin resistance state of their donors, using insulin AUC measurement as a proxy target variable for insulin resistance.

We first evaluated whether baseline clinical characteristics (age, sex, BMI, and fasting glucose) could be used to predict insulin resistance in the cohort in this study. We found the baseline parameters to be only moderately predictive (R²: 0.45; RMSE: 84.8) (FIG. 4B). We then trained a multiple linear regression model using the iPSC-EC gene expression profiles on NOS3, IRS1, and CDH13, and the resulting risk score closely matched measured insulin AUC(R²: 0.832; RMSE: 47.2) (FIG. 4C). The accuracy of prediction is further improved by replacing CDH13 with iPSC-EC NO release under stimulus (not shown) which closely matched empirically measured insulin AUC in the donors of the iPSC lines (R²: 0.855; RMSE: 43.8). Among the iPSC-EC gene expression and function parameters, NOS3 expression had the most prominent weight on predicting donor insulin AUC, and a variance analysis (FIG. 5B) provided evidence that inclusion of iPSC-ECs eNOS expression significantly improved upon prediction of insulin AUC over donor age, BMI, and fasting glucose alone (ANOVA Pr(>F): 0.06).

Although this model was constructed with a limited sample size, we attempted to validate the predictions using an independent cohort of patients with family history and current diagnosis of T2DM. Since insulin resistance is the driving force behind the pathogenesis of T2DM and insulin resistant or prediabetic patients progress to T2DM over time, we hypothesized that iPSC-EC derived from T2DM patients would have a similar molecular and cellular signature. The baseline demographics did not reveal whether the patients were diabetic with abnormal fasting glucose and HgbA1c (FIG. 4D). Similar to insulin resistant lines, the iPSC-EC lines had elevated NOS3 expression, decreased IRS1 expression and elevated CDH13 expression (FIG. 4E).

When using the gene expression algorithm with NOS3, IRS1, and CDH13, T2DM iPSC-EC had a profile that predicted an elevated insulin AUC (FIG. 4F). In our view, an advantage of using iPSC-EC for prediction is that because they are agnostic to epigenetic and environmental modifications. Risk assessment may be done in patients prior to onset of clinical symptoms; furthermore, the functional phenotypes of ECs we measured (eNOS expression and NO release) are physiologically proximal to pathological processes, and likely integrate the total summated output of many minute genetic risk modifiers and hence might be expected to present a clearer readout than polygenic risk score analysis.

Although the performance of the linear model would require validation in an independent cohort, the results here clearly support the feasibility of using patient-derived iPSC-EC characteristics to predict insulin-resistance status in the patient donors. In our view, an important advantage of using iPSC-EC for prediction is that because they are agnostic to epigenetic and environmental modifications, risk assessment may be done in patients prior to onset of clinical symptoms; furthermore, the functional phenotypes of ECs we measured (eNOS expression and NO release) are physiologically proximal to pathological processes, and likely integrate the total summated output of many minute genetic risk modifiers and hence might be expected to present a clearer readout than polygenic risk score analysis.

### Discussion

As the world becomes increasingly affluent and undergoes lifestyle changes, the continued rise of insulin resistance and diabetes will threaten to incur a considerable global health burden. Strikingly, South Asians have a higher prevalence of insulin resistance, diabetes and cardiovascular disease when compared to European age matched cohorts. Although variations in socioeconomic status and education could account for parts of these observations³¹, data from the U.K. with both European and South Asians exposed to the same Western diet and sedentary lifestyle indicate there is a genetic predisposition that can be captured in iPSC in vitro⁶.

The clinical diagnosis of insulin resistance is difficult with no evidence-based methodology. The euglycemic insulin clamp, the gold standard in research is difficult to perform in a clinical setting and bypasses the gastrointestinal intestinal L cells that secrete incretins that not only stimulate glucose disposal by increasing insulin secretion and decreasing glucagon activity but also improve insulin sensitivity 3. The oral glucose challenge is easier to perform in an outpatient clinical setting, but is time consuming and not as accurate 3. Without a high-throughput mechanism of testing, insulin resistance will remain undetected and is a missed opportunity to intervene and alter disease trajectory.

A risk scoring calculator based on iPSC-ECs is expected to be easier to use in a clinical setting than the current standard. Our data suggest that South Asians have abnormal gene expression and endothelial phenotypes at baseline. Thus, insulin-resistant patients would be identified before the onset of disease. Thus, early identification would allow individuals to modify their disease trajectory before the onset of endothelial dysfunction and subsequently atherosclerosis.

The baseline perturbations in gene expression are expected to be the consequence of SNPs. The identification and characterization of SNPs that cause insulin resistance and cardiovascular disease could lead to more accurate and precise risk calculators. CDH13 locus is over one million base pairs in size and contains multiple SNPs with appreciable allelic frequency. The clinical significance of most are unclear, but some SNPs are associated with insulin resistance, diabetes and cardiovascular disease. A risk scoring calculator based on SNPs from CDH13 validated by CRISPR cas9 mutagenesis would further augment the predictive power of the iPSC risk scoring calculator.

Genome-wide association studies have identified SNPs in multiple genes that contribute to insulin resistance and cardiovascular disease³⁴⁻³⁶. The mechanisms of polygenic disease are complex and difficult to reduce. The effect of each SNP and gene are likely small and have a syncretic effect on the phenotype. Moreover, the confounding effect of the environment makes it difficult to study the effects of genetics on insulin resistance and diabetes. iPSC-derived model systems can remove the effect of the environment with passaging—not unlike healthy diet and exercise can improve cardiovascular risk. Phenotypic differences in iPSC endothelial cells are likely due to SNPs and not the environment. Although there is considerable variability in iPSC disease modeling^{37,38}, we found that the expression of genes associated with cardiometabolic disease could predict the onset of insulin resistance the precursor of diabetes.

iPSC are an ideal system to correct SNPs and assess for amelioration in phenotype via genome editing. We have uncovered baseline differences in gene expression of NOS3, IRS1 and CDH13 along with NO production from South Asian insulin-resistant patient endothelial cells that are suggestive of an underlying genetic cause. The siRNA experiments suggest that CDH13 is over stimulating the insulin signaling cascade and causing increased NOS3 expression. Paradoxically eNOS is uncoupled and does not produce sufficient nitric oxide. In the setting of hyperglycemia and inflammation caused by central adiposity, the insulin signal cascade is further dampened and reactive oxygen species production is unchecked by NO. Further investigation of SNPs in CDH13 or the promoter region are warranted to determine if any of these mutations causes increased gene expression, NOS3 expression and uncoupling of nitric oxide production.

Currently, diet, exercise and metformin are the only evidence-based therapies for insulin resistance, which can also delay onset. Novel drug therapies are lacking but impaired NO production is the biochemical cause of endothelial dysfunction in the setting of hyperglycemia and increased ROS production. The imbalance in ROS and NO production results in damage to intracellular proteins and nucleic acid. A lack of NO causes smooth muscle proliferation, increased cell adhesion molecule expression that ultimately leads to macrophage recruitment and the accumulation of lipid plaque in the vascular wall. Statins effectively prevent cardiovascular events in type 2 diabetics. In South Asian insulin-resistant iPSC-ECs, statins appear to rescue nitric oxide production by improving NO production. Preclinical trials are currently underway to use statins in insulin resistance, but novel therapeutics are desperately needed to delay and possibly prevent the emergence of this imminent health care crisis.

### Methods

### Cell Culture:

The iPSC-ECs were cultured in EGM-2 media (Lonza, CC-3162). When confluent, iPSC-ECs were washed twice with phosphate-buffered saline (PBS), pH 7.4 and incubated in EGM-2 media without fetal bovine serum for 12 h. iPSC-ECs were treated with vehicle, 12.5 mM glucose solution (Invitrogen, A2494001), 5 ng/ml TNFα (R&D systems, 210-TA-020) or the combination of 12.5 mM glucose and 5 ng/ml TNFα. After incubation in conditioned media, iPSC-ECs were treated with 100 nM insulin (Sigma, 11061-68-0) for 20 minutes, washed with PBS twice and collected with TrypLE Express (Gibco, 12605036).

Cell Transfection with siRNA Experiments:

iPSC-ECs were seeded at a density of 2.5×10⁴/well in a 6-well plate in EGM-2 media. At 24 h post seeding, the cells were washed once with phosphate-buffered saline, pH 7.4, and replaced with 2 ml of OptiMEM media (Life Technologies, 31985-070). siRNA for CDH13 (Silencer™ Select Pre-Designed siRNA (siRNA ID: s2805), ThermoFisher, 4392420) or negative control (Silencer™ Select Negative Control No. 1 siRNA) was transfected using Lipofectamine™ RNAiMAX Transfection Reagent (ThermoFisher, 13778030). The cells were incubated for 4 h with a total of 10 ng siRNA or control RNA. Media was subsequently removed and replaced with EGM2 media (Lonza). After 48 h, RNA extracts were prepared using the RNEasy mini kit (Qiagen, 74104).

### Quantitative Real Time PCR

For RNA extraction and qRT-PCR of mRNA transcripts, total was isolated using the RNEasy mini kit (Qiagen, 74104). RNA was tested for purity (UV absorption ratio, A260/A280>1.9) and reverse transcribed using the High-Capacity cDNA Reverse Transcription Kit with RNase Inhibitor (ThermoFisher, 4374966). For qPCR, Taqman primers (Table 2) were employed with primers corresponding to the exact sequences of each individual mRNA. Samples were analyzed with technical triplicates. Primers were annealed at 55° C. and qPCR were performed on Biorad as per the manufacturer's instructions, and normalized to expression of the housekeeping genes GAPDH.

### Immunoblots:

After serum starvation and insulin treatment, iPSC-ECs were harvested with trypsin and pelleted by centrifugation. The pellet was washed with PBS and resuspended in Radio-immunoprecipitation assay (RIPA) buffer (ThermoFisher, 89900) with a Halt™ Phosphatase Inhibitor Cocktail (ThermoFisher, 78420). A cell scraper was used to collect lysate, which was transferred to a microcentrifuge tube. The samples were centrifuged 14,000×g for 15 minutes to pellet the cell debris. The cell extracts were normalized using BCA method and loaded on a 4-12% Bis-Tris Protein Gels, 1.5 mm, 10 well) (NuPAGE® Novex®, NP0335BOX) and subjected to electrophoresis for 90 min at 200 V. Proteins were transferred to PVDF membranes (Immun-Blot PVDF Membrane, Bio-Rad, 162-0174) and blocked in 5% BSA. The membrane was incubated at with primary antibody pan Akt (1:1000, Cell Signaling Technologies, 4691S), phospho-Akt (1:1000, Cell Signaling Technologies, #9271S), eNOS (1:1000, BD Bioscience, 610296), pS1117-eNOS (1:500, BD Bioscience, 612392) or GAPDH (1:5000 (ThermoFisher, MA5-15738-HRP) overnight at 4′C. The membranes were washed and incubated with goat anti-rabbit IgG (H+L)-HRP Conjugate (Bio-Rad, 1706515) for 1 h at RT. The membranes were again washed and then incubated with Clarity™ Western ECL Substrate (Bio-Rad, 1705060) for 5 min and detected on Bio-Rad Image Lab scanner. Densitometry was performed on Image Lab software (Bio-Rad).

### Angiogenesis Assay:

iPSC-ECs were treated serum free EGM-2 with the combination of 12.5 mM glucose (Invitrogen, A2494001) and 5 ng/ml TNFα (R&D systems, 210-TA-020). After incubation for 12 to 16 h, iPSC-ECs were treated with 100 nM insulin (Sigma, 11061-68-0) for 20 minutes, washed with PBS twice and collected with TrypLE Express (Gibco, 12605036). iPSC-ECs were resuspended in EGM-2 media and cells were counted. iPSC-ECs were plated in 24 well plate coated with BD MATRIGEL (Fisher Scientific, 08 774 552) at a density of 4-8×10⁴ cells per well. After incubation overnight, images were taken on a compound light microscope. Images were quantified for tube length and branch points using ImageJ software (Version 1.52, NIH).

### Nitric Oxide Assay:

iPSC-ECs were plated at a density of 2.5×10⁴ in 6 well plates or 5×10³ for 96 well plates. The iPSC-ECs were stimulated with 100 nM insulin for 20 min. The supernatants were collected and subjected to Nitrate/Nitrite Fluorometric Assay Kit (Cayman Chemical Co., 780051).

For the 4-amino-5-methylamino-2′,7′-difluorescein (DAF-FM) assay, iPSC-ECs were incubated with DAF-FM in serum free EGM-2 for 20 minutes with insulin. After washing twice in PBS, cells were incubated in PBS for an additional 20 minutes for de-esterification. Cells were harvested with TrypLE Express (Gibco, 12605036) and pelleted at 300 RCF at 4′C. After washing with PBS twice, the cells were resuspended in PBS with 2% BSA and analyzed with flow cytometry.

### Reactive Oxygen Species (ROS) Analysis:

iPSC-ECs were plated at a density of 5×10³ in 96 well plates in EGM2 media. iPSC-ECs were treated with vehicle, 12.5 mM glucose (Invitrogen, A2494001), 5 ng/ml TNFα (R&D systems, 210-TA-020) or 12.5 mM glucose and 5 ng/ml TNFα for 12 to 16 h. ROS activity was determined using the ROS-Glo™ H2O2 Assay (G8820) and analyzed on a GloMax® 96 Microplate Luminometer (Promega, GM3000)

### EndoPAT

Endothelial function was measured using the reactive hyperemia index (RHI) with an EndoPAT 2000 device (Itamar Medical, Israel)³⁹ A blood pressure cuff was placed on one arm and the contralateral arm served as the control. The study participants were seated in a quiet, dimly lit room with the temperature between at 25′C for a minimum of 5 minutes before measurements. After the equilibration period, the blood pressure cuff was inflated to 60 mmHg above systolic pressure or 200 mmHg and blood flow was occluded for 5 minutes. Cuff deflation induced reactive hyperemia. The RHI was calculated using (Endo-PAT2000 software version 3.0.4).

TABLE 1
Baseline characteristics of study participants in a. healthy control
and b. insulin resistant groups. In addition to the age, sex, BMI,
fasting glucose and insulin area under the curve, the cardiovascular
risk factors and family history are listed for study patients.
a

| | Healthy control | | | | |
|---|---|---|---|---|---|
| | HC1 | HC2 | HC3 | HC4 | HC5 |
| Age | 35 | 36 | 30 | 36 | 45 |
| Sex | F | M | M | M | F |
| BMI | | 25 | 24 | 22 | |
| SBP | | 112 | 133 | | |
| DBP | | 65 | 79 | | |
| Tobacco Use | − | − | − | − | − |
| HTN | − | − | − | − | − |
| Lipids | − | + | + | − | − |
| DM2 | − | − | − | − | − |
| FH DM | − | − | − | − | − |
| Total Cholesterol | | 235 | 241 | | |
| LDL | | 156 | 171 | | |
| HDL | | 48 | 56 | | |
| CDP | | | 0.8 | | |
| LP(a) | | 79 | 39.1 | | |
| Fasting Glucose | | 91 | 81.5 | 92 | |
| HOMA-IR | | 1.3 | 0.8 | | |
| Insulin AUC | | 75 | 4.75 | 88 | |

b

| | Insulin resistant | | | | |
|---|---|---|---|---|---|
| | IR1 | IR2 | IR3 | IR4 | IR5 |
| Age | 70 | 27 | 40 | 60 | 30 |
| Sex | M | M | M | M | M |
| BMI | 25 | 26 | 29 | 31 | 33 |
| SBP | 131 | 126 | 131 | 147 | 136 |
| DBP | 76 | 77 | 88 | 90 | 93 |
| Tobacco Use | − | − | + | − | − |
| HTN | − | − | + | + | − |
| Lipids | − | + | + | + | + |
| DM2 | − | − | − | − | − |
| FH DM | + | + | + | + | − |
| Total Cholesterol | 190 | 182 | 174 | 127 | 198 |
| LDL | 112 | 103 | 112 | 54 | 118 |
| HDL | 48 | 36 | 43 | 41 | 59 |
| CDP | 0.8 | 1.8 | | 1.3 | |
| LP(a) | 46.3 | 28.9 | | 19.6 | 58.2 |
| Fasting Glucose | 100 | 95 | 89 | 102 | 90 |
| HOMA-IR | 2.2 | 2.4 | 4.4 | 4.5 | 2.3 |
| Insulin AUC | 377 | 271 | 265 | 187 | 218 |

TABLE 2
Primers used for quantitative real time PCR.

| Primer List | |
|---|---|
| Primer | Catalog number |
| GAPDH | Hs02758991_g1 |
| NOS3 | Hs01574659_m |
| CHD13 | Hs01004531_m |
| IRS1 | Hs00178563_m |
| SPRED1 | Hs01084559_m |
| SIRT1 | Hs01009006_m |
| ICAM1 | Hs00164932_m |
| VCAM1 | Hs01003372_m |
| CD31 | Hs00169777_m |
| CD5 | Hs00901465_m |

### REFERENCES

1. Reaven, G. M. Banting lecture 1988. Role of insulin resistance in human disease. Diabetes 37, 1595-1607 (1988).
2. Malik, S. et al. Impact of the metabolic syndrome on mortality from coronary heart disease, cardiovascular disease, and all causes in United States adults. Circulation 110, 1245-1250 (2004).
3. Reaven, G. Insulin resistance and coronary heart disease in nondiabetic individuals. Arterioscler. Thromb. Vasc. Biol. 32, 1754-1759 (2012).
4. Forouhi, N. G., Sattar, N., Tillin, T., McKeigue, P. M. & Chaturvedi, N. Do known risk factors explain the higher coronary heart disease mortality in South Asian compared with European men? Prospective follow-up of the Southall and Brent studies, UK. Diabetologia 49, 2580-2588 (2006).
5. Dhawan, J., Bray, C. L., Warburton, R., Ghambhir, D. S. & Morris, J. Insulin resistance, high prevalence of diabetes, and cardiovascular risk in immigrant Asians. Genetic or environmental effect? Br. Heart J. 72, 413-421 (1994).
6. McKeigue, P. M., Ferrie, J. E., Pierpoint, T. & Marmot, M. G. Association of early-onset coronary heart disease in South Asian men with glucose intolerance and hyperinsulinemia. Circulation 87, 152-161 (1993).
7. Donner, C. C. et al. Presentation of a new method for specific measurement of in vivo insulin-stimulated glucose disposal in humans: comparison of this approach with the insulin clamp and minimal model techniques. J. Clin. Endocrinol. Metab. 60, 723-726 (1985).
8. Meigs, J. B. Epidemiology of the insulin resistance syndrome. Curr. Diab. Rep. 3, 73-79 (2003).
9. Li, Z. et al. Functional and transcriptional characterization of human embryonic stem cell-derived endothelial cells for treatment of myocardial infarction. PLoS One 4, e8443 (2009).
10. Williams, I. M. & Wu, J. C. Generation of Endothelial Cells From Human Pluripotent Stem Cells: Methods, Considerations, and Applications. Arterioscler. Thromb. Vasc. Biol. 39, 1317-1329 (2019).
11. Hu, S. et al. Effects of cellular origin on differentiation of human induced pluripotent stem cell-derived endothelial cells. JCI Insight 1, (2016).
12. Stumvoll, M. et al. Use of the oral glucose tolerance test to assess insulin release and insulin sensitivity. Diabetes Care 23, 295-301 (2000).
13. Ross, R. Atherosclerosis—an inflammatory disease. N. Engl. J. Med. 340, 115-126 (1999).
14. Matsa, E. et al. Transcriptome Profiling of Patient-Specific Human iPSC-Cardiomyocytes Predicts Individual Drug Safety and Efficacy Responses In Vitro. Cell Stem Cell vol. 19 311-325 (2016).
15. Sharma, A. et al. High-throughput screening of tyrosine kinase inhibitor cardiotoxicity with human induced pluripotent stem cells. Science Translational Medicine vol. 9 eaaf2584 (2017).
16. Paik, D. T. et al. Large-Scale Single-Cell RNA-Seq Reveals Molecular Signatures of Heterogeneous Populations of Human Induced Pluripotent Stem Cell-Derived Endothelial Cells. Circ. Res. 123, 443-450 (2018).
17. Hotamisligil, G. S., Arner, P., Caro, J. F., Atkinson, R. L. & Spiegelman, B. M. Increased adipose tissue expression of tumor necrosis factor-alpha in human obesity and insulin resistance. J. Clin. Invest. 95, 2409-2415 (1995).
18. Shoelson, S. E., Lee, J. & Goldfine, A. B. Inflammation and insulin resistance. J. Clin. Invest. 116, 1793-1801 (2006).
19. Sjöholm, A. & Nystrom, T. Endothelial inflammation in insulin resistance. Lancet 365, 610-612 (2005).
20. Verma, S. et al. A self-fulfilling prophecy: C-reactive protein attenuates nitric oxide production and inhibits angiogenesis. Circulation 106, 913-919 (2002).
21. Li, G., Barrett, E. J., Barrett, M. O., Cao, W. & Liu, Z. Tumor necrosis factor-alpha induces insulin resistance in endothelial cells via a p38 mitogen-activated protein kinase-dependent pathway. Endocrinology 148, 3356-3363 (2007).
22. Kuboki, K. et al. Regulation of endothelial constitutive nitric oxide synthase gene expression in endothelial cells and in vivo: a specific vascular action of insulin. Circulation 101, 676-681 (2000).
23. Mattagajasingh, I. et al. SIRT1 promotes endothelium-dependent vascular relaxation by activating endothelial nitric oxide synthase. Proc. Natl. Acad. Sci. U.S.A 104, 14855-14860 (2007).
24. Nathan, C. Nitric oxide as a secretory product of mammalian cells. FASEB J. 6, 3051-3064 (1992).
25. Nims, R. W. et al. Colorimetric Methods for the Determination of Nitric Oxide Concentration in Neutral Aqueous Solutions. Methods 7, 48-54 (1995).
26. Green, L. C. et al. Analysis of nitrate, nitrite, and [15N] nitrate in biological fluids. Anal. Biochem. 126, 131-138 (1982).
27. King, G. L. & Johnson, S. M. Receptor-mediated transport of insulin across endothelial cells. Science 227, 1583-1586 (1985).
28. Kitamoto, A. et al. CDH13 Polymorphisms are Associated with Adiponectin Levels and Metabolic Syndrome Traits Independently of Visceral Fat Mass. J. Atheroscler. Thromb. 23, 309-319 (2016).
29. Philippova, M. et al. T-cadherin attenuates insulin-dependent signalling, eNOS activation, and angiogenesis in vascular endothelial cells. Cardiovasc. Res. 93, 498-507 (2012).
30. Laufs, U., La Fata, V., Plutzky, J. & Liao, J. K. Upregulation of endothelial nitric oxide synthase by HMG CoA reductase inhibitors. Circulation 97, 1129-1135 (1998).
31. Yusuf, S. et al. Cardiovascular risk and events in 17 low-, middle-, and high-income countries. N. Engl. J. Med. 371, 818-827 (2014).
32. Wu, S. et al. The effects of incretin-based therapies on β-cell function and insulin resistance in type 2 diabetes: A systematic review and network meta-analysis combining 360 trials. Diabetes Obes. Metab. (2018) doi:10.1111/dom.13613.
33. Borai, A., Livingstone, C. & Ferns, G. A. A. The biochemical assessment of insulin resistance. Ann. Clin. Biochem. 44, 324-342 (2007).
34. Qi, L. et al. Genetic susceptibility to coronary heart disease in type 2 diabetes: 3 independent studies. J. Am. Coll. Cardiol. 58, 2675-2682 (2011).
35. Dauriz, M. et al. Association of a 62 Variants Type 2 Diabetes Genetic Risk Score With Markers of Subclinical AtherosclerosisCLINICAL PERSPECTIVE. Circulation: Genomic and Precision Medicine 8, 507-515 (2015).
36. LeBlanc, M. et al. Identifying Novel Gene Variants in Coronary Artery Disease and Shared Genes with Several Cardiovascular Risk Factors. Circ. Res. 118, 83-94 (2016).
37. Carcamo-Orive, I., Huang, N. F., Quertermous, T. & Knowles, J. W. Induced Pluripotent Stem Cell-Derived Endothelial Cells in Insulin Resistance and Metabolic Syndrome. Arterioscler. Thromb. Vasc. Biol. 37, 2038-2042 (2017).
38. Carcamo-Orive, I. et al. Analysis of Transcriptional Variability in a Large Human iPSC Library Reveals Genetic and Non-genetic Determinants of Heterogeneity. Cell Stem Cell 20, 518-532.e9 (2017).
39. Bonetti, P. O. et al. Enhanced external counterpulsation improves endothelial function in patients with symptomatic coronary artery disease. J. Am. Coll. Cardiol. 41, 1761-1768 (2003).

1. An in vitro method of determining whether a subject is at risk of developing insulin resistance, the method comprising:
a) obtaining a biological sample comprising somatic cells from the subject;
b) generating induced pluripotent stem cells (IPSCs) from the somatic cells;
c) differentiating the IPSCs into endothelial cells (IPSC-ECs);
d) measuring one or more biomarkers in the IPSC-ECs derived from the subject, wherein the biomarkers are selected from the group consisting of phosphorylated Akt, reactive oxygen species (ROS), nitric oxide (NO), SIRT1, eNOS, CDH13, and IRS1; and
e) determining whether the subject is at risk of developing insulin resistance, wherein reduced phosphorylation of Akt, increased levels of ROS, decreased levels of expression of SIRT1 and IRS1, and increased levels of expression of eNOS and CDH13 in the IPSC-ECs derived from the subject compared to reference values ranges for levels of the biomarkers in control IPSC-ECs indicate the subject is at risk of developing insulin resistance.

2. The method of claim 1, further comprising administering a pre-diabetic treatment if the subject is determined to be at risk of developing insulin resistance.

3. The method of claim 2, wherein the pre-diabetic treatment comprises administering a therapeutically effective amount of metformin to the subject, modifying diet of the subject to reduce carbohydrate intake, reducing weight of the subject, increasing amount of exercise of the subject, administering medication to treat high blood pressure in the subject, administering medication to treat high cholesterol in the subject, ceasing smoking of the subject, or a combination thereof.

4. The method of claim 3, wherein treatment further comprises administering to the subject a sulfonylurea, a thiazolidinedione, a dipeptidyl peptidase-4 inhibitor, a sodium-glucose transport protein 2 (SGLT2) inhibitor, a glucagon-like peptide-1 analog, an angiotensin-converting enzyme inhibitor (ACEI), or a combination thereof.

5-6. (canceled)

7. The method of claim 1, wherein the subject has one or more risk factors for developing insulin resistance or type 2 diabetes selected from the group consisting of obesity, a sedentary lifestyle, a genetic predisposition for developing diabetes, a family history indicating a risk of developing diabetes, polycystic ovary syndrome, hepatitis C, high blood pressure, high cholesterol, age of 45 or older, history of gestational diabetes, vitamin D deficiency, treatment with HIV drugs having protease inhibitors, or treatment with growth hormone replacement therapy.

8. The method of claim 1, wherein the somatic cells are peripheral blood mononuclear cells (PBMCs), fibroblasts, keratinocytes, epithelial cells, or endothelial progenitor cells.

9. The method of claim 1, wherein said measuring the levels of expression of a biomarker selected from the group consisting of SIRT1, eNOS, CDH13, and IRS1 comprises measuring a level of a mRNA or a protein.

10-12. (canceled)

13. The method of claim 1, wherein the subject has not yet developed clinical symptoms.

14. (canceled)

15. The method of claim 1, further comprising stimulating the IPSC-ECs with glucose and/or tumor necrosis factor-alpha (TNFα) before said measuring the one or more biomarkers in the IPSC-ECs derived from the subject.

16. A method of monitoring progression of prediabetes in a subject, the method comprising:
a) obtaining a first biological sample comprising somatic cells from the subject at a first time point and a second biological sample comprising somatic cells from the subject later at a second time point;
b) generating induced pluripotent stem cells (IPSCs) from the somatic cells in the first biological sample and the second biological sample;
c) differentiating the IPSCs derived from the somatic cells of the first biological sample and the second biological sample into endothelial cells (IPSC-ECs);
d) optionally stimulating the IPSC-EC with a cocktail containing high glucose and/or TNFα to simulate the hyperglycemic and inflammatory milieu found in prediabetic patients prior to biomarker measurements;
e) measuring one or more biomarkers in the IPSC-ECs derived from the somatic cells of the first biological sample and the second biological sample, wherein the biomarkers are selected from the group consisting of phosphorylated Akt, reactive oxygen species (ROS), nitric oxide (NO), SIRT1, eNOS, CDH13, IRS1 and NO production; and
f) evaluating progression of prediabetes in the subject wherein detection of reduced phosphorylation of Akt, increased levels of ROS, decreased levels of expression of SIRT1 and IRS1, and increased levels of expression of eNOS and CDH13 in the IPSC-ECs derived from the somatic cells of the second biological sample compared to the IPSC-ECs derived from the somatic cells of the first biological sample indicates that the risk of the subject developing insulin resistance is increasing, and detection of increased phosphorylation of Akt, decreased levels of ROS, increased levels of expression of SIRT1 and IRS1, and decreased levels of expression of eNOS and CDH13 in the IPSC-ECs derived from the somatic cells of the second biological sample compared to the IPSC-ECs derived from the somatic cells of the first biological sample indicates that the risk of the subject developing insulin resistance is decreasing.

17. The method of claim 16, wherein the subject has developed clinical symptoms.

18. (canceled)

19. The method of claim 16, wherein the somatic cells are peripheral blood mononuclear cells (PBMCs), fibroblasts, keratinocytes, epithelial cells, or endothelial progenitor cells.

20. The method of claim 16, further comprising stimulating the IPSC-ECs derived from the somatic cells of the first biological sample and the second biological sample with glucose and/or TNF-α before said measuring the one or more biomarkers.

21. A method of monitoring efficacy of a pre-diabetic treatment in a subject, the method comprising:
a) obtaining a first biological sample comprising somatic cells from the subject before the subject undergoes the pre-diabetic treatment and a second biological sample comprising somatic cells from the subject after the subject undergoes the pre-diabetic treatment;
b) generating induced pluripotent stem cells (IPSCs) from the somatic cells in the first biological sample and the second biological sample;
c) differentiating the IPSCs derived from the somatic cells of the first biological sample and the second biological sample into endothelial cells (IPSC-ECs);
d) measuring one or more biomarkers in the IPSC-ECs derived from the somatic cells of the first biological sample and the second biological sample, wherein the biomarkers are selected from the group consisting of phosphorylated Akt, reactive oxygen species (ROS), nitric oxide (NO), SIRT1, eNOS, CDH13, IRS1 and NO production;
e) evaluating the efficacy of the prediabetic treatment, wherein detection of reduced phosphorylation of Akt, increased levels of ROS, decreased levels of expression of SIRT1 and IRS1, and increased levels of expression of eNOS and CDH13 in the IPSC-ECs derived from the somatic cells of the second biological sample compared to the IPSC-ECs derived from the somatic cells of the first biological sample indicates that the subject is worsening or not responding to the pre-diabetic treatment, and detection of increased phosphorylation of Akt, decreased levels of ROS, decreased level of NO production, increased levels of expression of SIRT1 and IRS1, and decreased levels of expression of eNOS and CDH13 in the IPSC-ECs derived from the somatic cells of the second biological sample compared to the IPSC-ECs derived from the somatic cells of the first biological sample indicates that the subject is improving; and
f) combining one or more biomarker measurements into a predictive statistical or machine learning model to calculate the risk score of an individual for developing insulin resistance

22. The method of claim 21, wherein the subject has developed clinical symptoms.

23. The method of claim 21, wherein the pre-diabetic treatment comprises administering a therapeutically effective amount of metformin to the subject, modifying diet of the subject to reduce carbohydrate intake, reducing weight of the subject, increasing amount of exercise of the subject, bariatric surgery, administering medication to treat high blood pressure in the subject, administering medication to treat high cholesterol in the subject, ceasing smoking of the subject, or a combination thereof.

24. The method of claim 23, wherein treatment further comprises administering to the subject a sulfonylurea, a thiazolidinedione, a dipeptidyl peptidase-4 inhibitor, a sodium-glucose transport protein 2 (SGLT2) inhibitor, a glucagon-like peptide-1 analog, an angiotensin-converting enzyme inhibitor (ACEI), or a combination thereof.

25-27. (canceled)

28. The method of claim 21, wherein the somatic cells are peripheral blood mononuclear cells (PBMCs), fibroblasts, keratinocytes, epithelial cells, or endothelial progenitor cells.

29. The method of claim 21, further comprising stimulating the IPSC-ECs derived from the somatic cells of the first biological sample and the second biological sample with glucose or TNF-α before said measuring the one or more biomarkers.

30-33. (canceled)

34. A model of insulin resistance comprising an induced pluripotent stem cell-derived endothelial cell (IPSC-EC), wherein the IPSC-EC is derived from a somatic cell of a subject and treated with tumor necrosis factor-alpha (TNFα), interleukin-1 beta (IL1-β), or interferon gamma (INFγ).
